(19) United States
(12) Patent Application Publication
(10) Pub. No.: US 2016/0045629 A1
(43) Pub. Date: Feb. 18, 2016

(54) Disinfectant Caps

(71) Applicant: EXCELSIOR MEDICAL CORPORATION, Neptune, NJ (US)

(72) Inventors: Christopher E. Gardner, Manalapan, NJ (US); Robert Banik, Edgewater, NJ (US)

(73) Assignee: EXCELSIOR MEDICAL CORPORATION, Neptune, NJ (US)

(21) Appl. No.: 14/826,180

(22) Filed: Aug. 13, 2015

Related U.S. Application Data
(60) Provisional application No. 62/036,666, filed on Aug. 13, 2014.

(51) Int. Cl. A61L 2/18

(52) U.S. Cl. CPC A61L 2/18

(57) ABSTRACT

Disinfectant caps and packaging for use with a medical device (e.g., connector, luer access device, etc.) are disclosed herein. The disinfectant caps apply the antiseptic fluid (e.g., disinfectant) directly onto the surface of the medical device. The disinfectant cap for disinfecting a surface of a medical device comprises a cap body having a bottom wall and a sidewall extending therefrom, the sidewall defining a top opening, antiseptic fluid contained within the cap body, and a deformable top wall connected to the cap body, the top wall movable from a first position sealing the antiseptic fluid within the cap body to a second position releasing the antiseptic fluid from the cap body.

### CROSS-REFERENCE TO RELATED APPLICATIONS

This application claims the benefit of U.S. Provisional Application No. 62/036,666 filed on Aug. 13, 2014, the entire disclosure of which is expressly incorporated herein by reference.

### BACKGROUND

1. Field of the Disclosure

The present invention relates to disinfectant caps for medical devices. More specifically, the present invention relates to disinfectant caps for luer access devices that provide direct contact with antiseptic fluid stored therein.

2. Background

Intravenous (IV) devices are widely used to administer fluids to patients, such as through the use of a catheter inserted into a patient. Usually, the catheter is connected to an injection site, such as a luer access device, which provides fluid communication from a fluid source (e.g., IV bag, syringe, etc.) to the patient. The connectors are frequently separated from each other (e.g., when a patient needs to use the bathroom), which exposes the connectors to the environment, which can result in contamination.

To reduce the risk of contamination, the connectors are usually disinfected between uses. Current procedures include swabbing the connectors with a disinfecting pad, which is prone to human error and not often implemented. Alternatively, antiseptic caps are used to clean and cover the connectors. However, many antiseptic caps require a presoaked absorbent material (e.g., absorbent pad, absorbent sponge, etc.) inserted therein to store and subsequently release the antiseptic fluid onto the connector (or any other medical device). Furthermore, the injection sites typically utilize a male luer thread geometry to facilitate connection of syringes and IV tubing for fluid communication. Existing antiseptic caps utilize the corresponding female luer thread geometry to secure the cap to the injection site. However without the additional tapered luer tip geometry, which secures these types of connections between syringes and injection sites, the antiseptic caps do not securely fit on the injection site, and are prone to falling off inadvertently.

### SUMMARY

The present invention relates to disinfectant caps and packaging for use with a medical device (e.g., connector, luer access device, etc.). The disinfectant caps apply the antiseptic fluid (e.g., disinfectant) directly onto the surface of the medical device. The disinfectant caps incorporate specific thread geometry to provide a secure fit to threaded access sites.

### BRIEF DESCRIPTION OF THE DRAWINGS

The foregoing features of the disclosure will be apparent from the following Detailed Description, taken in connection with the accompanying drawings, in which:

FIG. 1 is a perspective view of a disinfectant cap with an invertible wall;

FIG. 2a-2c illustrate steps for manufacturing the disinfectant cap of FIG. 1;

FIG. 3a-3b illustrate steps for applying the disinfectant cap of FIG. 1 to a luer access device;

FIG. 4 is a side view of another disinfectant cap with a bulb;

FIG. 5 is a cross-sectional view of a disinfectant cap with an elastomeric dome cover;

FIG. 6 is a cross-sectional view of the disinfectant cap of FIG. 5 engaged with a luer access device;

FIG. 7 is a perspective view of an elastomeric dome cover with ribs;

FIG. 8 is a cross-sectional view of the elastomeric dome cover of FIG. 7;

FIG. 9 is a perspective view of the elastomeric dome cover of FIG. 7 inverted;

FIG. 10 is a cross-sectional view of the elastomeric dome cover of FIG. 7 inverted and attached to a cap body of a disinfectant cap;

FIG. 11 is a perspective view of an elastomeric dome cover with a helical rib;

FIG. 12 is a cross-sectional view of the elastomeric dome cover of FIG. 11 inverted;

FIG. 13 is a perspective view of a disinfectant cap with an elastomeric dome insert;

FIG. 14 is a cross-sectional view of the disinfectant cap of FIG. 13;

FIG. 15 is a cross-sectional view of the disinfectant cap of FIG. 13 engaged with a luer access device;

FIG. 16 is a cross-sectional view of a disinfectant cap with a movable plug;

FIG. 17 is a cross-sectional view of the disinfectant cap of FIG. 16 engaged with a luer access device;

FIG. 18 is a cross-sectional view of a disinfectant cap with a threadable insert;

FIG. 19 is a perspective view of a disinfectant cap with inner variable threads;

FIG. 20 is a side view of the disinfectant cap of FIG. 19;

FIG. 21 is a cross-sectional view of the disinfectant cap of FIG. 19;

FIG. 22 is a perspective view of a disinfectant cap with inner variable thread segments;

FIG. 23 is a side view of the disinfectant cap of FIG. 22;

FIG. 24 is a cross-sectional view of the disinfectant cap of FIG. 22;

FIG. 25 is a top view of the disinfectant cap of FIG. 22 with a pre-soaked absorbent material inserted therein;

FIG. 26 is a cross-sectional view of the disinfectant cap of FIG. 25 with the pre-soaked absorbent material inserted therein;

FIGS. 27A-27C are perspective views of a disinfectant cap sealed by a film with a scored area;

FIGS. 28A-28C are perspective views of a disinfectant cap with a notch for receiving an attached area of a film attached to a disinfectant cap;

FIG. 29 is a cross-sectional perspective view of a disinfectant cap with a dome insert sealed in a cap holder;

FIG. 30 is a cross-sectional side view of the disinfectant cap and cap holder of FIG. 29;

FIG. 31 is a cross-sectional side view of a disinfectant cap with a dome insert in a cap holder, the disinfectant cap having a bottom opening;

FIG. 32 is an exploded cross-sectional perspective view of the disinfectant cap and cap holder of FIG. 31;

FIGS. 33A-33B are cross-sectional side views illustrating use of the disinfectant cap and cap holder of FIG. 31;

FIG. 34 is a cross-sectional side view of a disinfectant cap with an integrally formed internal dome barrier in a cap holder, the disinfectant cap having a bottom opening;

FIG. 35 is an exploded cross-sectional perspective view of the disinfectant cap and cap holder of FIG. 34;

FIG. 36 is a cross-sectional view of a disinfectant cap with a bottom plug;

FIG. 37 is a perspective view of a disinfectant cap with a frangible neck;

FIG. 38 is a cross-sectional perspective view of the disinfectant cap of FIG. 37;

FIG. 39 is a perspective view of a disinfectant cap with a frangible neck and angled fingers;

FIG. 40 is a cross-sectional perspective view of the disinfectant cap of FIG. 39; and

FIGS. 41A-41C are cross-sectional side views illustrating use of the disinfectant cap of FIG. 39 and cap holder.

### DETAILED DESCRIPTION

The present disclosure relates to disinfectant caps for disinfecting a luer access device (LAD) or any other medical device. More specifically, the disclosure relates to disinfectant caps with antiseptic fluid (e.g., alcohol, isopropyl alcohol, etc.) to disinfect a surface of an LAD. Many of the disinfectant caps of the present disclosure do not require an absorbent material (e.g., an absorbent sponge or pad). The disinfectant caps described below could be manufactured using any suitable technique (e.g., blow molding, injection molding, etc.), and using one or more of a variety of materials (e.g., polypropylene, polyethylene, etc.). The disinfectant cap could be applied to the medical device regardless of the orientation of the medical device. The features described with respect to a particular embodiment could be used with other embodiments described herein.

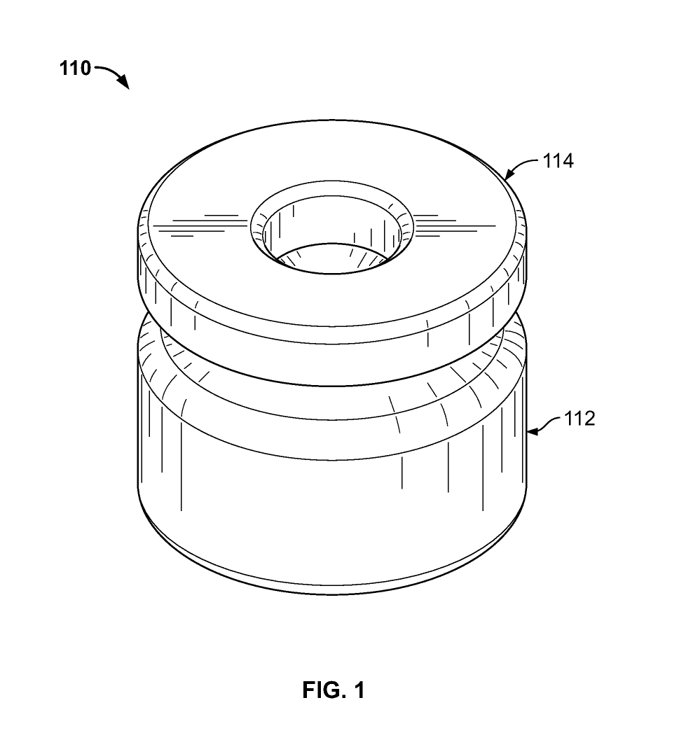

FIGS. 1-3 show a disinfectant cap 110 with an invertible wall. More specifically, FIG. 1 is a perspective view of a disinfectant cap 110 with an invertible wall. The disinfectant cap includes a cap body 112 and a cover 114 attached thereto. The cap body 112 could be made out of a rigid or semi-rigid plastic (e.g., polypropylene, polyethylene, etc.) or other suitable material. The cap may be manufactured by a variety of methods including blow molding, injection molding, stamping, vacuum forming, etc.

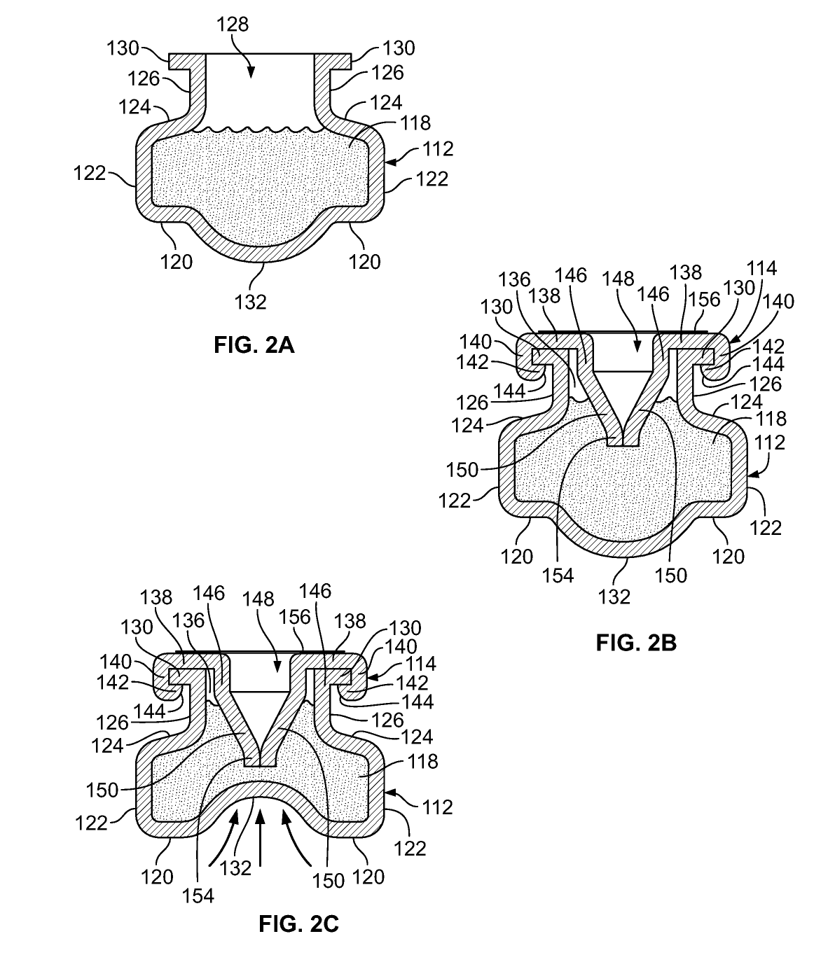

FIG. 2a-2c illustrate steps for manufacturing the disinfectant cap of FIG. 1. As shown, in FIG. 2a, the cap body 112 is filled with antiseptic fluid 118 (e.g., alcohol, isopropyl alcohol, ethanol, hydrogen peroxide, povidone iodine, Chlorhexidine Gluconate, triclosan, etc.). The cap body 112 includes a bottom wall 120, a sidewall 122 extending therefrom, which could include one or more angled segments, such as angled wall 124 (e.g., tapered or horizontal), and a neck 126 extending from sidewall 122, which all define an interior. The inner circumference of the neck 126 can be smaller than the circumference of the sidewall 122. The neck 126 defines a top opening 128 providing access to the interior of the cap body 112, and can include a flange 130 extending (e.g., perpendicularly) from the top of the neck 126. The bottom wall 120 of the cap body 112 can include an invertible wall 132 which is in a first orientation convex (e.g., outwardly bulging). Although shown as positioned on the bottom wall 120, the invertible wall 132 could be located anywhere on the cap body 112 (e.g., formed in the sidewall 122).

In FIG. 2b, a cover 114 is attached to the top of the cap body 112 (e.g., by snapping on, pressing gluing, over-molding, ultrasonically welding, etc.), thereby sealing the antiseptic fluid 118 therein. The cover 114 could be made of a hard plastic. Once attached and sealed, the cover 114 and the cap body 112 define an interior having a first volume, and the antiseptic fluid 118 and gas 136 (e.g., air or atmosphere), if any, are stored therein at a first pressure (e.g., atmospheric pressure).

The cover 114 can include a planar circular top portion 138 with a depending rim 140 extending downwardly from a peripheral edge of the planar circular top portion 138. The depending rim 140 can include a lip 142 extending (e.g., perpendicularly) from a bottom thereof. The lip 142 could include an annular rounded edge 144 (or taper) to facilitate application of the cover 114 to the cap body 112. The lip 142 of the rim 140 engages the flange 130 of the neck 126 of the cap body 112 to secure the cover 114 to the cap body 112. However, any form of attachment could be used (e.g., friction fit, adhesion, welding, overmolding, etc.).

The cover 114 can include an inner wall 146 extending downwardly from the top portion 138 of the cover 114 and defining a central aperture 148. The inner wall 146 can include a portion of reducing size 150 (e.g., tapered, conical, etc.) extending downwardly to a frangible tip 154 that extends into the interior of the cap body 112. The inner walls 146 can be made of a hard (e.g., rigid, semi-rigid, etc.) plastic and the frangible tip 154 forms a breakable nozzle. Alternatively, the inner walls 146 can be made of a soft material (e.g., rubber, low durometer material, silicone or thermoplastic) and the tip 154 can be slit or manufactured with a small opening (e.g., hole or slit). The inner walls 146 and the tapered portion 150 are sized and shaped to engage a medical implement that requires disinfecting, such as a luer access device (not shown). When the cover 114 is attached to the cap body 112, the frangible tip 154 may be submerged in the antiseptic fluid 118 contained in the cap body 112. A removable film 156 (e.g., paper peel, peelable sterile barrier, foil lidstock, etc.) could be adhered (or otherwise attached) to the top surface of the cover 114, such as over the central aperture 148.

It is noted that the cap body 112 does not require a neck 126 (e.g., the sidewall 122 defines a top opening of the cap body 112), and that any shape or type of cap body 112 and/or cover 114 could be used. The cover 114 could include threads (or other mating feature such as protrusions, recesses, and/or snap fits) molded into the inner walls 146 (such as in the tapered portion 150) to mate with and retain the LAD. Alternatively, the inner walls 146 could be sized to form an interference fit with the LAD. The inner walls 146 could be made of a material of sufficient softness to allow threads on the LAD to penetrate into the surface of the inner walls 146 so as to create mating female threads on the smooth surface.

In FIG. 2c, after the disinfectant cap 110 is filled and sealed, pressure can be exerted (e.g., by a user, by a machine) upon the invertible wall 132 of the bottom wall 120 until the invertible wall 132 deforms inwardly to a second concave orientation. The inward deformation (e.g., deflection) of the invertible wall 132 decreases the volume of the interior defined by the cover 114 and the cap body 112, thereby increasing the pressure within the disinfectant cap 110 (e.g., so that the pressure is greater than atmospheric pressure).

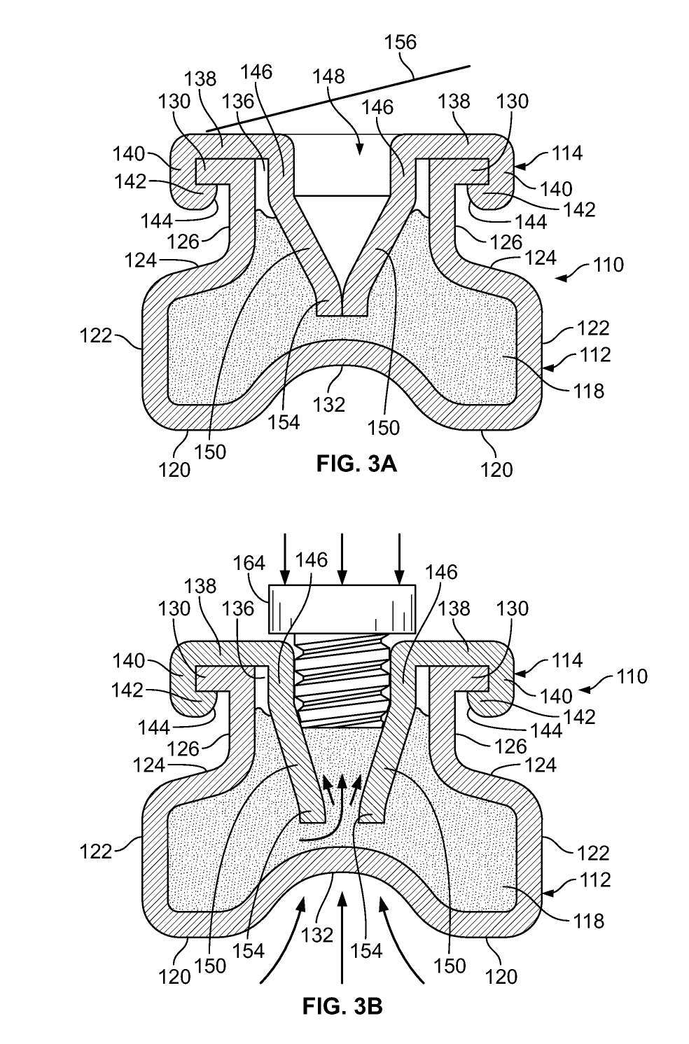

FIG. 3a-3b illustrate steps for applying the disinfectant cap 110 of FIG. 1 to a luer access device. In FIG. 3a, a user removes the film 156 adhered to the top of the cover 114, thereby providing access to the central aperture 148 defined by the cover 114. In FIG. 3b, the disinfectant cap 110 is applied to an LAD 164 (or other medical device) by inserting (e.g., pushing, threading, etc.) the LAD 164 into the central aperture 148. The tapered portion 150 of the cover 114 may be smaller in size (e.g., diameter) than the LAD 164. In this way, when the LAD 164 is inserted into the aperture 148, the frangible tip 154 of the tapered portion 150 breaks, so that the LAD 164 is wedged between the tapered portion 150 and fully engaged with the disinfectant cap 110. Once the frangible tip 154 breaks the pressurized antiseptic fluid 118 sprays out onto the surface of the LAD 164 (because the pressure within the disinfectant cap 110 is greater than atmospheric pressure) and fills the area around the LAD 164, thereby disinfecting the LAD 164. Alternatively, the invertible wall 132 could be deformed after application of the disinfectant cap 110 to the LAD 164. Deforming the invertible wall after application of the disinfectant cap to the LAD may be preferable if the frangible tip 154 is replaced by a slit or other small opening. In this configuration the disinfectant may not be forced through the opening upon removal of the film. Rather, upon insertion of the LAD, the opening at the tip 154 becomes larger in size to allow greater fluid flow from the cavity 118 onto the LAD. The initial size of the slit or opening at the tip 154 can be sized to minimize the amount of disinfectant that might leak through the tip 154 if the cap 120 is positioned upside down prior to insertion of the LAD. For example, a small hole or slit may be prone to contain the disinfectant as a liquid tends not to flow through a small hole unless pressurized (e.g., due to liquid surface tension).

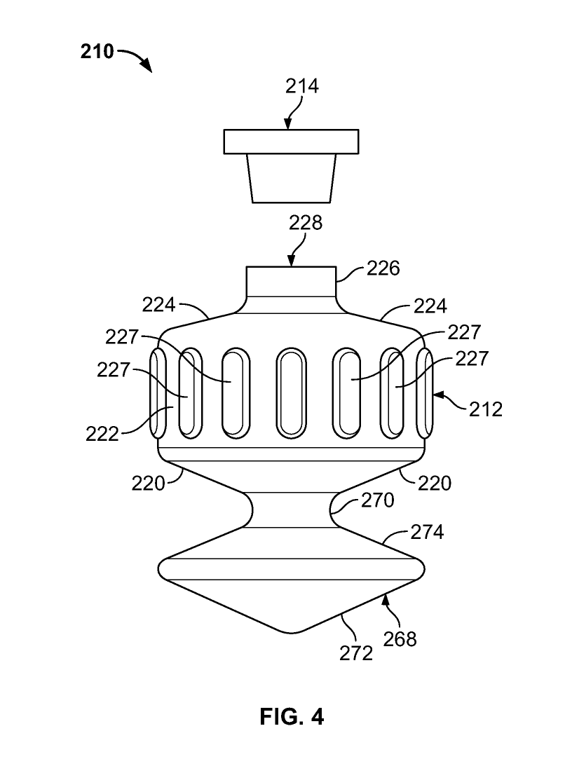

FIG. 4 is a side view of another disinfectant cap 210 with a bulb. More specifically, the disinfectant cap 210 includes a cap body 212 and a cover 214. The cap body 212 includes a bottom wall 220, a sidewall 222 which could include one or more angled segments such as angled wall 224, and a neck 226, all of which define an interior, as described above. The outer surface of the sidewall 222 could include a plurality of annularly spaced vertical ribs 266 oriented along the long central axis of the disinfectant cap 210. The vertical ribs 227 facilitate gripping and twisting of the disinfectant cap 210 by a user.

The inner circumference of the neck 226 is smaller than the circumference of the sidewall 222. The neck 226 defines a top opening 228 providing access to the interior of the cap body 212. A cover 214 (e.g., breakable seal insert) is inserted into the neck 226 to seal the interior of the disinfectant cap 210.

The bottom wall 220 of the cap body 212 includes a bulb 268 integrally formed with (or attached to) the cap body by a stem 270. The circumference of the bulb 268 could be sized similarly to, or smaller than, the circumference of the cap body 212. The bulb 268 defines an interior, which is in fluid communication with the interior of the cap body 212 via a channel within the stem 270.

The bottom surface 272 (and/or top surface 274) of the bulb 268 could be invertible to decrease the volume of the disinfectant cap 210 and increase the pressure of the antiseptic fluid and any gas contained therein. The bulb 268 could be deformed before or after engagement with an LAD, such as by squeezing the bulb 268 to direct disinfecting fluid through the stem 270 and onto an LAD or other medical device. The bulb 268 can be designed such that it retains its deformed shape to maintain internal pressure. For example, the bottom surface 272 (e.g., bottom wall) of the bulb 268 could be designed with a slight outward bow so that as pressure is exerted the bottom surface 272 eventually reaches a point of inflection where it flexes and inverts, and then remains nested in the top surface 274 (e.g., similar to a locking bellow).

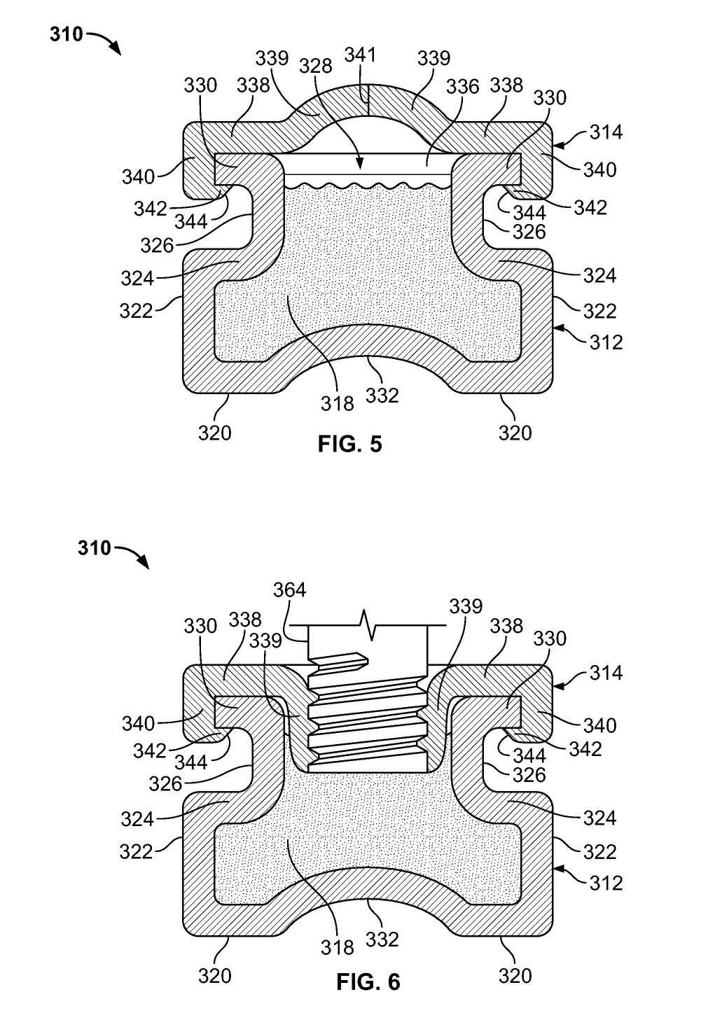

FIGS. 5-12 show disinfectant caps with domed covers. More specifically, FIG. 5 is a cross-sectional view of a disinfectant cap 310 with an elastomeric dome cover 314. The disinfectant cap 310 of FIGS. 5-12 includes a cap body 312, which includes a bottom wall 320, a sidewall 322 which could include one or more angled segments such as angled wall 324, and a neck 326, all of which define an interior, as described above. The neck 326 defines a top opening 328 providing access to the interior of the cap body 312, and includes an outwardly extending flange 330 extending from the top of the neck 326. The disinfectant cap 310 shown in FIG. 5 is fully assembled and manufactured such that the bottom wall 320 of the cap body 312 includes an invertible wall 332, but the invertible wall is not required and the bottom wall 320 could be of any shape and contour.

The elastomeric dome cover 314 includes a generally planar outer periphery 338 with a centrally located dome portion 339. A depending rim 340 extends downwardly from the outer periphery 338 a peripheral edge of the outer periphery 338. The rim 340 include a lip 342 extending from a bottom of the rim 340. As described above, the lip 342 of the rim 340 engages the flange 330 of the neck 326 of the cap body 312 to secure the elastomeric dome cover 314 to the cap body 312. However, any form of attachment could be used (e.g., friction fit, adhesion, etc.).

The elastomeric dome cover 314 includes a dome portion 339 (e.g., hemispherical wall) having one or more partial slits 341 formed through (or near) the apex thereof. The elastomeric dome cover 314 is attached to the top of the cap body 312, thereby retaining antiseptic fluid 318 and gas 336, if any, therein. Alternatively, the disinfectant cap 310 could be filled with antiseptic fluid 318 through the partial slits 341. Further, instead of partial slits 341, the elastomeric dome cover 314 could have a weakened area made by other means (e.g., a thin wall that separates when pressure is applied to the cover or when the cover is inverted). Instead of partial slits 341, the elastomeric dome cover 314 could have a small hole which retains the disinfectant in the cavity through the surface tension of the liquid. The elastomeric dome cover 314 (e.g., and partial slits 341 or small hole) could be sealed with a lidstock.

The elastomeric dome cover 314 and the cap body 312 define an interior having a first volume and the antiseptic fluid 318 and gas 336 (e.g., air or atmosphere), if any, is stored therein at a first pressure (e.g., atmospheric pressure). A removable film could be applied (e.g., adhered) to the top surface of the elastomeric dome cover 314.

FIG. 6 is a cross-sectional view of the disinfectant cap 310 of FIG. 5 engaged with a luer access device 364. To apply the disinfectant cap 310 to an LAD 364, a user removes the film (if any) from the top surface of the elastomeric dome cover 314. The disinfectant cap 310 is then applied to the LAD 364 (or other medical device) so that the LAD 364 contacts and then begins to compress and deform the elastomeric dome cover 314 such that the elastomeric dome cover 314 deforms inwardly, which decreases the volume of the interior of the disinfectant cap 310 and increases the pressure of the antiseptic fluid 318 and any gas 336 therein. The elastomeric dome cover 314 continues to deform and invert until the slit 341 at the apex of the dome portion 339 opens and pressurized antiseptic fluid 318 sprays on the surface of the LAD 364. The LAD 364 continues to engage the disinfectant cap 310 until it is frictionally secured to the inwardly deformed dome portion 339 of the cover 314 (e.g., the LAD 364 enters the inverted dome cover 314) and/or through the opened slits 341 of the dome cover 314. The elastomeric dome cover 314 stretches around the LAD 364, thereby securing itself to the LAD 364 (which eliminates the need for threads) and sealing the antiseptic fluid 318 between the dome cover 314 and the LAD 364. Alternately, the inner surface of the cap body 312 (e.g., sidewall 322, neck 326, etc.) could incorporate threads, snaps, detents, or other engagement features to more securely attach the cap 310 to the LAD after the elastomeric dome cover 314 has been penetrated.

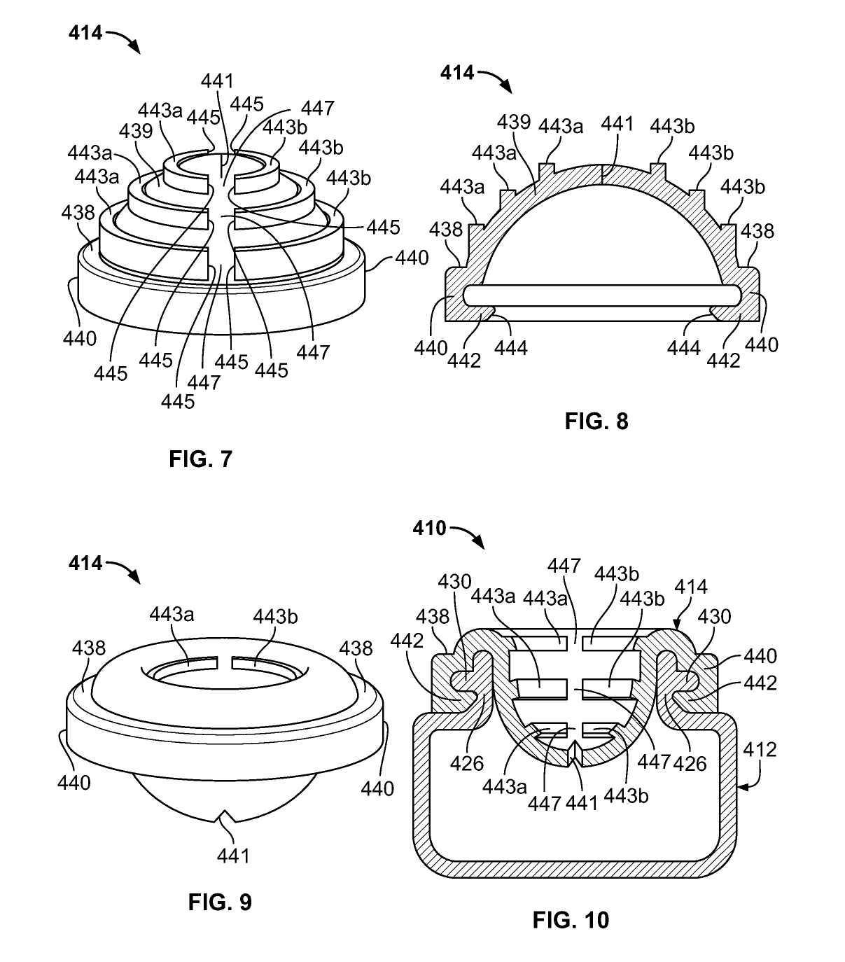

FIGS. 7-10 show an elastomeric dome cover 414 with ribs 443 that extend around the surface of the cover 414. The elastomeric dome cover 414 includes a planar circular top edge portion 438 with a centrally located dome 439. A rim 440 extends downwardly from a peripheral edge of the planar circular top edge portion 438. The rim 440 include an inwardly extending lip 442 extending from a bottom of the rim 440. The dome 439 has one or more partial slits 441 (and/or weakened area) formed through (or near) the apex thereof. The dome cover 414 can include concentric, outwardly extending, ribs 443 (e.g., circumferential rings). Although ribs 443 are described, other types of outwardly extending protrusions could be used.

The ribs 443 could extend vertically or horizontally or radially from the dome 439. The ribs 443 are non-continuous with aligned opposite ends 445 such that each rib 443 is composed of two rib segments (e.g., a first rib segment 443a and a second rib segment 443b). The breaks 447 between the rib segments 443 facilitate inversion of the elastomeric dome cover 414 by allowing the dome 439 to bend/flex. Although two rib segments 443 are shown, any number of rib segments 443 could be used, or a single non-continuous rib (e.g., with only one break).

FIG. 8 is a cross-sectional view of the elastomeric dome cover 414 of FIG. 7. As shown, the rim 440 includes a lip 442 extending from a bottom of the rim 440. An annular groove could be formed in the rim 440 to receive the cap body 412. The lip 442 could include an annular taper 444 to facilitate application of the elastomeric dome cover 414 to the cap body 412.

FIG. 9 is a perspective view of the elastomeric dome cover 412 of FIG. 7 inverted. As shown, the slit 441 of the elastomeric dome cover 412 is naturally forced open by inversion of the elastomeric dome cover 412.

FIG. 10 is a cross-sectional view of the elastomeric dome cover 414 of FIG. 7 inverted and attached to a cap body 412 of a disinfectant cap 410. The lip 442 of the rim 440 engages the flange 430 of the neck 426 of the cap body 412 to secure the elastomeric dome cover 414 to the cap body 412. When the elastomeric dome cover 414 is inverted the ribs 443 extend inwardly to facilitate engagement with the LAD (not shown).

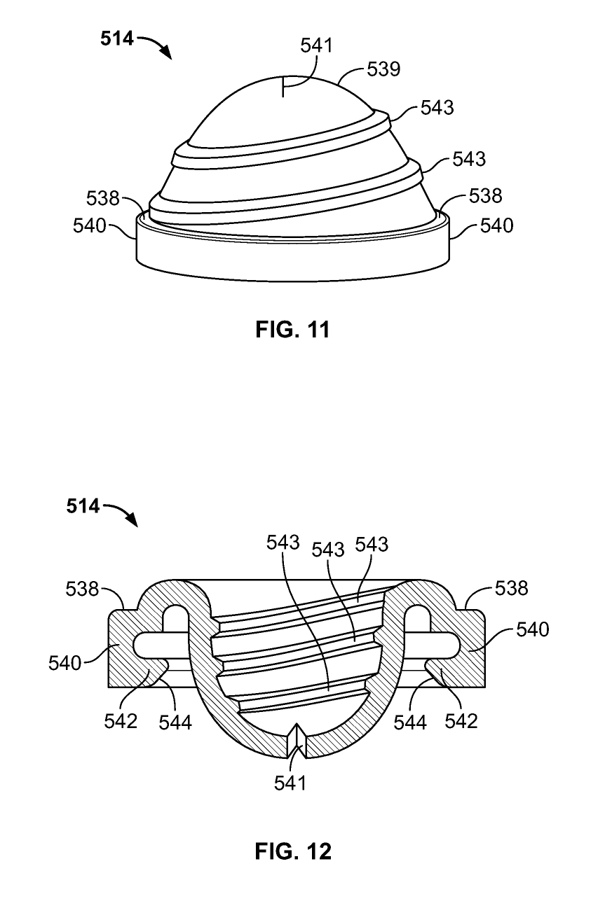

FIGS. 11-12 show another elastomeric dome cover 514 with a helical rib 543. More specifically, FIG. 11 is a perspective view of an elastomeric dome cover 514 with an outwardly extending helical rib 543 (e.g., spiral rib). The helical rib 543 could be continuous or non-continuous (the helical rib 543 could include one or more breaks). The elastomeric dome cover 514, as previously described, includes a planar circular top edge portion 538 with a centrally located dome 539. A depending rim 540 extends downwardly from a peripheral edge of the planar circular top edge portion 538. The dome 539 has one or more partial slits 541 (and/or weakened area) formed through (or near) the apex thereof.

FIG. 12 is a cross-sectional view of the elastomeric dome cover 514 of FIG. 11 inverted. As shown, the rim 540 includes a lip 542 extending from a bottom of the rim 540. An annular groove could be formed in the rim 540 to receive the cap body 512. The lip 452 includes an annular taper 544 to facilitate application of the elastomeric dome cover 514 to the cap body. As shown, the slit 541 of the elastomeric dome cover 514 is forced open when inverted. When the elastomeric dome cover 514 is inverted the helical rib 543 extends inwardly to facilitate engagement with the LAD (not shown). In this way, the helical rib 543 can form threads to engage the threads of the LAD.

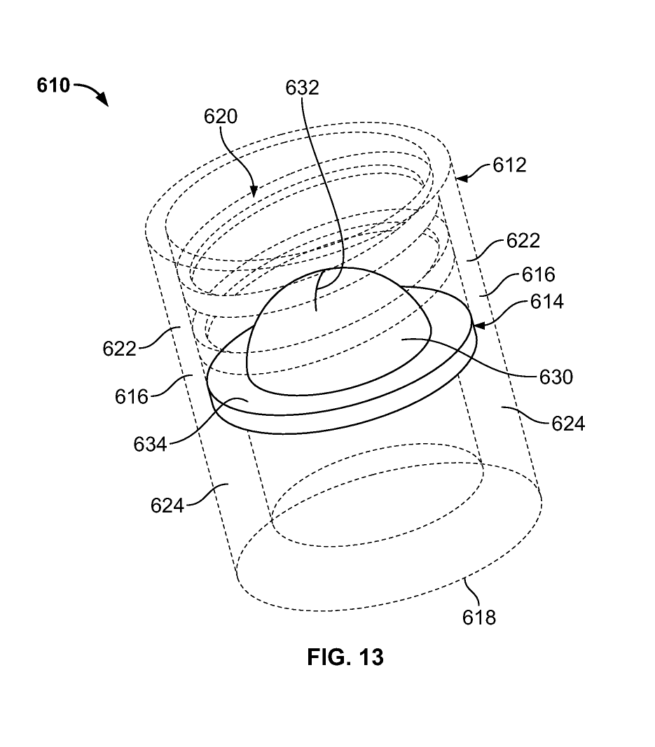

FIGS. 13-15 show a disinfectant cap 610 with an elastomeric dome insert 614. More specifically, FIG. 13 is a perspective view of a dome insert 614 in a disinfectant cap 610 (shown in dashed lines). The elastomeric dome insert 614 is positioned within the interior of the cap body 612. The elastomeric dome insert 614 includes a dome 630 (e.g., hemispherical wall) having one or more slits 632 (and/or weakened areas) formed through (or substantially near) the apex thereof. The elastomeric dome insert 614 further includes an annular disc 634 extending outwardly from the base of the dome 630.

As shown, the disinfectant cap 610 includes a cap body 612 with a sidewall 616 and a bottom wall 618 defining an interior. The top of the sidewall 616 defines a top opening 620. Although not shown, a removable film could be provided over the top opening 620. The sidewall 616 includes an upper portion 622 and a lower portion 624, with a shoulder (e.g., ledge) 626 therebetween, such that the wall of the upper portion 622 has a smaller thickness than the wall of the lower portion 624. The annular disc 634 rests on the shoulder (e.g., ledge) 626 of the cap body 612 when positioned therein.

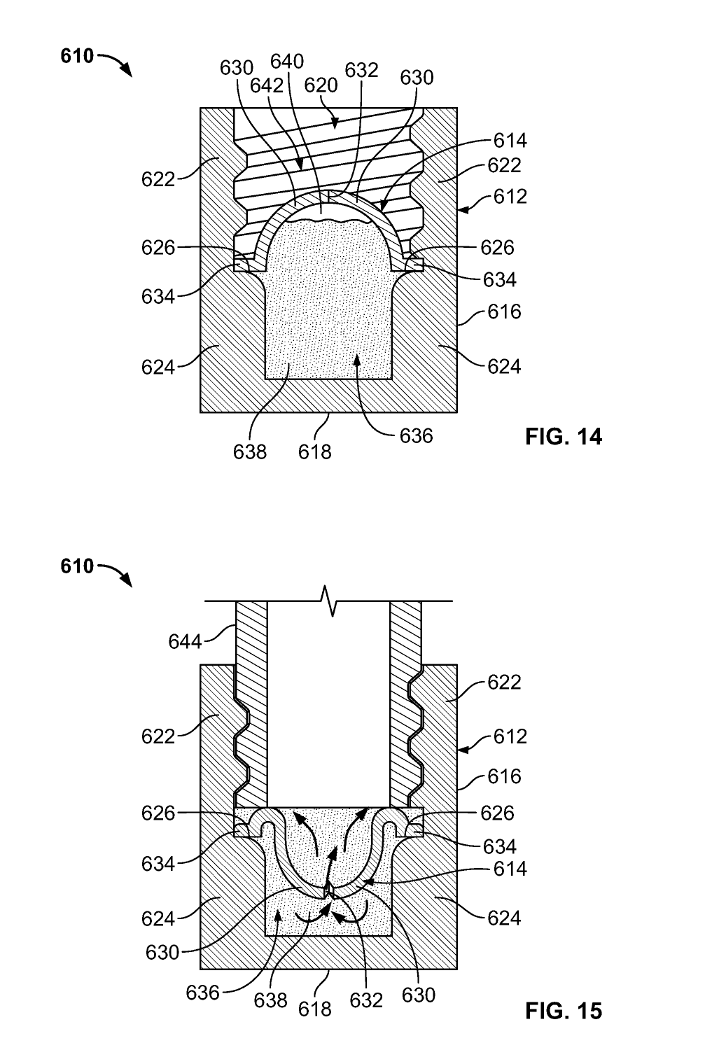

FIG. 14 is a cross-sectional view of the disinfectant cap 610 of FIG. 13. The lower portion 624 of the sidewall 616 of the cap body 612 and the bottom surface of the elastomeric dome insert 614 define a first chamber 636 for containing antiseptic fluid 638, and any gas or air 640, therein at a first volume and a first pressure. The antiseptic fluid 638 could fill the interior of the disinfectant cap such that the fluid level rises above the ledge 626 of the cap body 612.

The upper portion 622 of the sidewall 616 of the cap body 612 and the top surface of the elastomeric dome insert 614 define a second chamber 642 for receiving and engaging an LAD (not shown). The inner surface of the upper portion 622 of the sidewall 616 could be threaded and/or elastically deformable to engage the LAD. Alternate engagement features or mechanisms could be employed such as press fits, snap fits, grooves, recesses, etc.

FIG. 15 is a cross-sectional view of the disinfectant cap of FIG. 13 engaged with a luer access device 644. As shown, the disinfectant cap 610 threadably engages the LAD 644, until the LAD 644 makes contact with the elastomeric dome insert 614. As the LAD 644 continues to engage the disinfectant cap 610, the LAD 644 begins to deform the elastomeric dome insert 614 such that it deforms inwardly, which decreases the volume of the first chamber 638 and increases the pressure of the antiseptic fluid 638 and gas 640, if any, therein. The elastomeric dome insert 614 continues to deform until the slit 632 at the apex of the dome 630 opens and pressurized antiseptic fluid 638 sprays onto the surface of the LAD 644. In lieu of a slit 632, the elastomeric dome insert 614 could employ a small hole that allows the disinfectant liquid to pass through it when the dome is depressed by the LAD. The hole could be sized to retain the disinfectant liquid within the first chamber 636 (e.g., due to the liquid surface tension). The elastomeric dome insert 614 can be secured to the cap 612 by any means including snap fit, adhesion, bonding, ultrasonic welding, press fit, etc. Alternately, the cap 612 and elastomeric dome insert 614 can be made of a single component (e.g., integrally formed together).

It may be desirable to design the cap to allow evaporation of the disinfectant after application of the cap to the LAD. A significant residual pool of disinfectant can remain on the access site and increase the risk of infusing the disinfectant into the patient (in the example of disinfecting an LAD). Without any absorbent material present to remove residual disinfectant, it may be beneficial to allow excess disinfectant to evaporate. This can be accomplished by designing channels, slits, holes, gaps in the threading, or other means through which the disinfectant can evaporate to the external atmosphere.

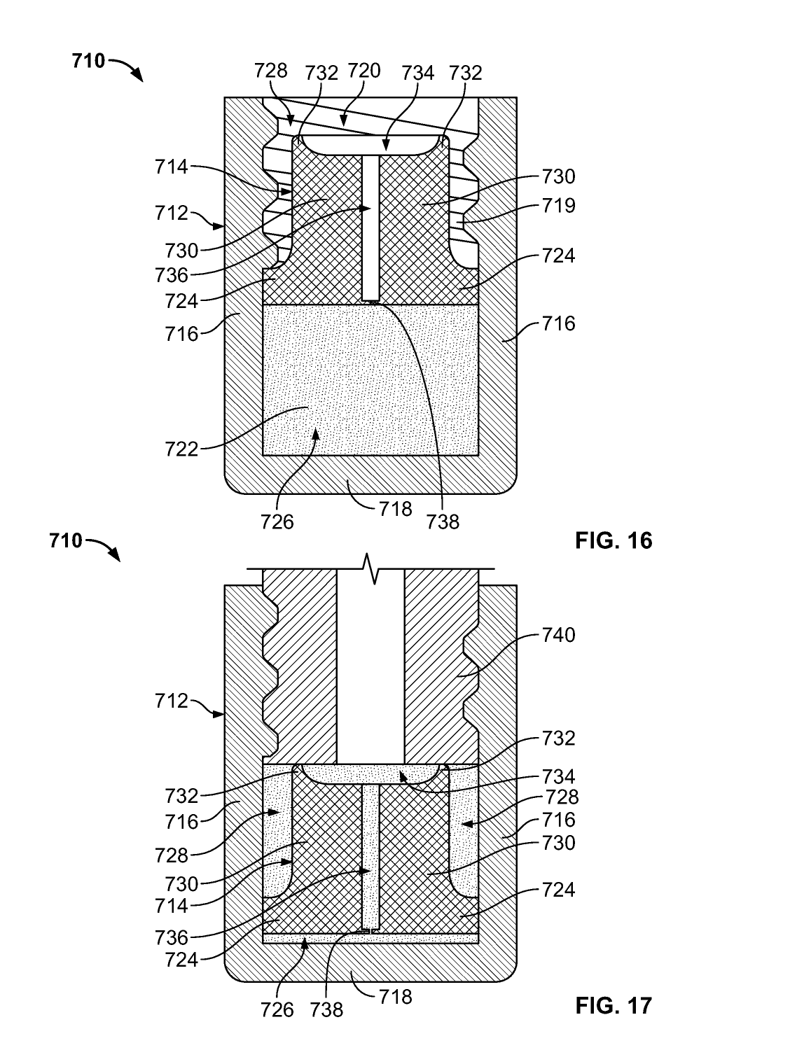

FIGS. 16-17 show a disinfectant cap 710 with a movable plug 714. More specifically, FIG. 16 is a cross-sectional view of a disinfectant cap 710 with a movable plug 714. As shown the disinfectant cap 710 includes a cap body 712 with a sidewall 716 and a bottom wall 718 defining an interior. The top of the sidewall 716 defines a top opening 720 which received the LAD (not shown). Although not shown, a removable film could be provided over the top opening 720. The inner surface of the sidewall 716 could be threaded 719 and/or deformable to engage the LAD.

Antiseptic fluid 722 is inserted into and contained within the cap body 712. A movable plug 714 (e.g., wall, divider, etc.) is positioned within the interior of the cap body 712 at or above the antiseptic fluid 722. The movable plug 714 includes a cylindrical bottom end 724 which has approximately the same size as the interior diameter of the cap body 712, and could form a friction fit with the cap body 712, or an o-ring could be added around the periphery of the bottom end 724 to prevent the antiseptic fluid 722 from seeping past the movable plug 714. The bottom surface of the bottom end 724 of the movable plug 714, the sidewalls 716, and the bottom wall 718 define a first chamber 726. The top surface of the movable plug 714 and the sidewalls 716 of the cap body 712 define a second chamber 728.

The movable plug further comprises a recessed section 730 extending from the bottom end 724. The upper portion of the movable plug 714 includes an annular rim 732, which defines a recess 734 therein and the annular rim 732 could be continuous or non-continuous (e.g., could include one or more breaks), and/or have one or more scallops in a top surface thereof. The recessed section 730 has a diameter that is smaller than that of the bottom end 724, to facilitate movement of the plug 714 does not interfere with the threads. Alternatively, instead of the recessed section 730, the rim 732 could simply extend from the top of the bottom end 724 (so that the entire plug 714 has one continuous sidewall of a substantially consistent diameter).

The recessed section 730 defines a vertical channel 736 extending from a top of the recessed section 730 through (or nearly through) a bottom of the bottom end 724. At the bottom of the channel 736 there could be an access point 738 (e.g., small hole, fluid seal, valve, or a frangible element built into (or attached to) the bottom end 724 of the plug 714 that prevents fluid from seeping into the channel 736. For example, the movable plug 714 may be made of a rigid material (e.g., polypropylene, polyethylene, etc.) with an access point 738 being a weaker area molded into the bottom end 724 of the plug 714, or the movable plug 714 could be an elastomer (e.g., silicone) with the access point 738 being a valve (e.g., check valve, duck-bill valve, thin slit valve, etc.) or small hole molded into the bottom end 724 of the plug 714.

FIG. 17 is a cross-sectional view of the disinfectant cap 710 of FIG. 16 engaged with a luer access device 740. As shown, the disinfectant cap 710 threadably engages (e.g., is placed onto) the LAD 740, until the LAD 740 makes contact with the annular rim 732 of the movable plug 714. As the LAD 740 continues to engage the disinfectant cap 710, the LAD 740 forces the movable plug 714 to translate into the interior of the cap body 712, which increases the pressure of the antiseptic fluid 722. The increased pressure forces the antiseptic fluid 722 through the access point 738 (e.g., breaks the frangible element, overcomes the retaining force of the valve, overcomes the liquid surface tension to pass through the hole, cracks the valve, etc.), so that antiseptic fluid 722 is then forced upward through the vertical channel 736 (e.g., the fluid 722 is displaced by advancing the plug 714 into the disinfectant cap 710). Once at the top of the vertical channel 736, the antiseptic fluid 722 then flows onto the surface of the LAD 740 and/or pools in the recess 734 defined by the rim 732 until overflowing into the space defined by the exterior surface of the recessed section 730 and the interior surface of the cap body 712. Once sufficiently engaged, the pooled antiseptic fluid 722 continuously contacts the surface of the LAD 740. In this way the antiseptic fluid 722 moves from the first chamber 726 to the second chamber 728, which submerges the plug 714.

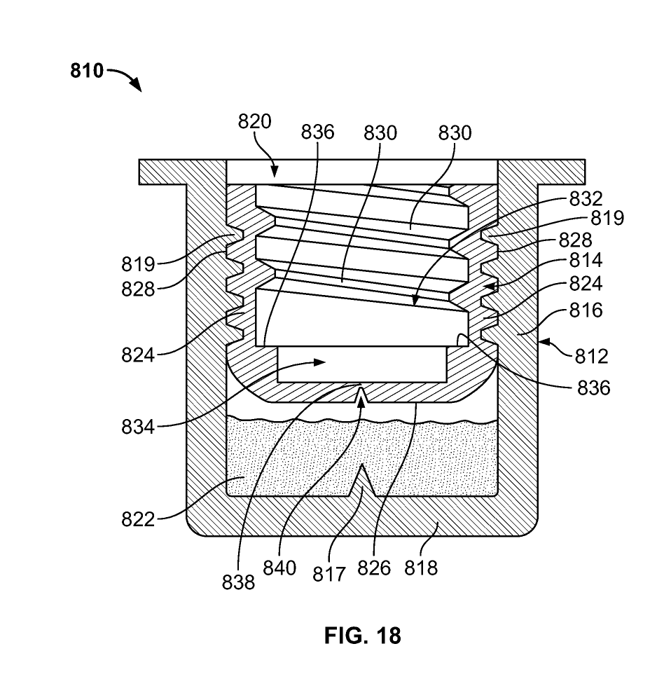

FIG. 18 is a cross-sectional view of a disinfectant cap 810 with a threadable insert 814 (e.g., movable plug). As shown the disinfectant cap 810 includes a cap body 812 with a sidewall 816 and a bottom wall 818 defining an interior. The top of the sidewall 816 defines a top opening 820 which receives the LAD (not shown). Although not shown, a removable film could be provided over the top opening 820. The inner surface of the sidewall 816 could be threaded 819 and/or deformable to engage the LAD. A conical spike 817 (e.g., sharp protrusion) extends inwardly from an (approximate) center of an interior surface of the bottom wall 818.

Antiseptic fluid 822 is inserted into and contained within the cap body 812. A threadable insert 814 is positioned within the interior of the cap body 812 at, or above, the antiseptic fluid 822 (thereby retaining the antiseptic fluid 822 in the cap body 812). The threadable insert 814 includes a sidewall 824 and a bottom wall 826 defining an interior. The threadable insert 814 includes outer threads 828 defined by an exterior surface of the sidewall 824, which engage the threads 819 of the sidewall 816 of the cap body 812, and could provide a seal therebetween. The threadable insert 814 also includes inner threads 830 defined by an interior surface of the sidewall 824, which engage the threads of the LAD (not shown). Alternatively, the threadable insert 814 could utilize a friction fit (instead of the outer threads 828 and/or inner threads 830).

The sidewall 824 of the threadable insert 814 includes an upper portion 832 and a lower portion 834, where the sidewall 824 of the upper portion 832 is thinner than that of the lower portion 834, thereby defining an annular ledge 836. The bottom wall 826 includes an access point 838 (e.g., frangible element and/or fluid seal) in the center of the bottom wall 818. Further, the access point 838 could define a conical recess 840 in the exterior surface thereof, where the conical recess 840 is positioned directly in line with (and could be correspondingly shaped to) the spike 817.

To apply the disinfectant cap 810 to the LAD, the disinfectant cap 810 is threaded onto the LAD, so that the threadable insert 814 moves relative to the LAD. Once the LAD bottoms out and contacts the ledge 836 of the threadable insert 814 (e.g., the LAD cannot engage the threadable insert any more), the threadable insert 814 stops moving relative to the LAD, and instead moves relative to the cap body 812. The disinfectant cap 810 is continued to be threaded and move laterally into the interior of the cap body 812, pressure could build by the decreased volume between the bottom wall 826 of the threadable insert 814 and the bottom wall 818 of the cap body 812. The cap 810 continues to move inwardly until the spike 817 of the cap body 812 punctures the access point 838 of the bottom wall 826 of the threadable insert 814. Once punctured, the pressurized antiseptic fluid 822 flows onto the surface of the LAD and pools in the lower portion 834 of the threadable insert 814 so that the antiseptic fluid 822 makes continuous contact with the LAD. The conical recess 840 of the bottom wall 826 of the threadable insert 814 delays the puncturing of the access point 838, which further decreases the volume (increasing the pressure) of the antiseptic fluid 822 and gas, if any.

Notably, the spike 817 of the disinfectant cap of FIG. 18 is not required and could be replaced with a valve or frangible element in the bottom wall of the threadable insert 814 (which breaks upon sufficient pressure), as described with respect to FIGS. 16-17. In this respect, the disinfectant cap of FIG. 16-17 could utilize the spike 817 discussed with the disinfectant cap 810 of FIG. 18. Alternately, the threadable insert 814 could have a hole in the bottom wall 826, which is sized to prevent the disinfectant fluid 822 from flowing therethrough (e.g., due to liquid surface tension), until the disinfectant fluid 822 is pressurized by threading of the threadable insert 814 into the disinfectant cap 810.

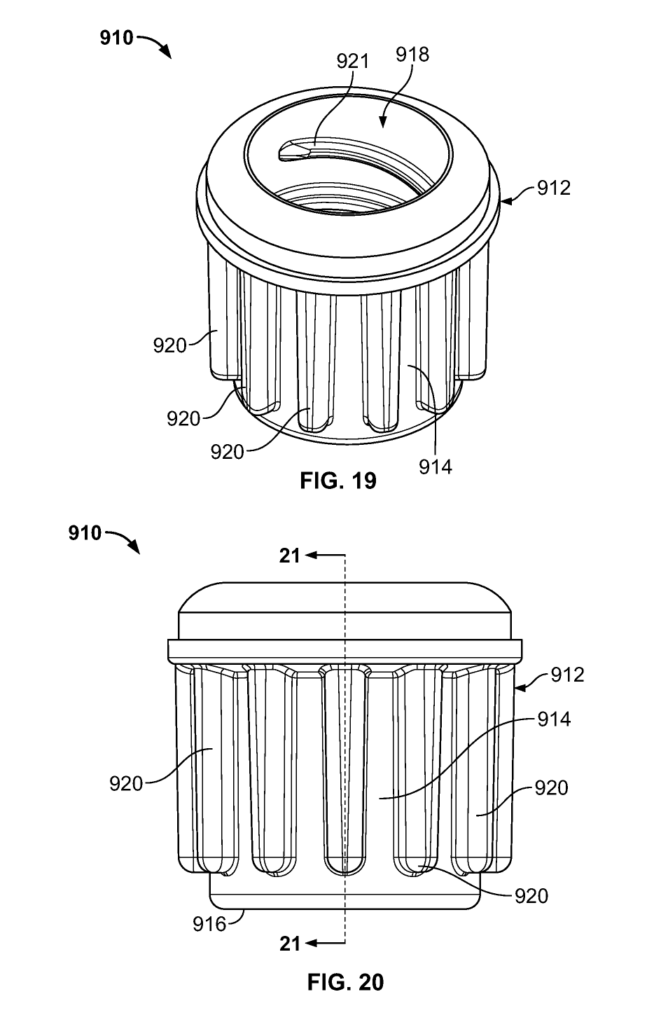

FIGS. 19-21 are views of a disinfectant cap 910 with inner variable threads 921. More specifically, FIG. 19 is a perspective view of a disinfectant cap 910 with inner variable threads 921, and FIG. 20 is a side view of the disinfectant cap of FIG. 19. Although variable threads are discussed with respect to a disinfectant cap, the variable threads could be used for any threaded medical device (e.g., any threaded luer access device). Further, the variable threads shown could be used with any of the embodiments discussed above.

The disinfectant cap 910 includes a cap body 912 with a sidewall 914 and a bottom wall 916 defining an interior. The top of the sidewall 914 defines a top opening 918. Although not shown, a removable film could be provided over the top opening 918. The outer surface of the sidewall 916 could include a plurality of annularly spaced vertical ribs 920 oriented along the axis of the disinfectant cap 910. The vertical ribs 920 facilitate gripping and twisting of the disinfectant cap 910 by a user. The inner surface of the sidewall 914 includes one or more variable threads 921. The disinfectant cap 910 can be stored in a second component, which can serve as an applicator and/or as packaging. The vertical ribs 920 can provide rotational lock between the disinfectant cap 910 and the second component (e.g., applicator). Lidstock can be applied to the second component (e.g., applicator) to provide a fully sterile packaging for the disinfectant cap 910. In applications where the cap 910 is stored within a second component, the lidstock can be adhered to both the cap 910 and the second component to provide two distinct chambers. Alternately, the cap can 910 be detached from the lidstock such that it is open within the chamber that is formed by the second component and the lidstock.

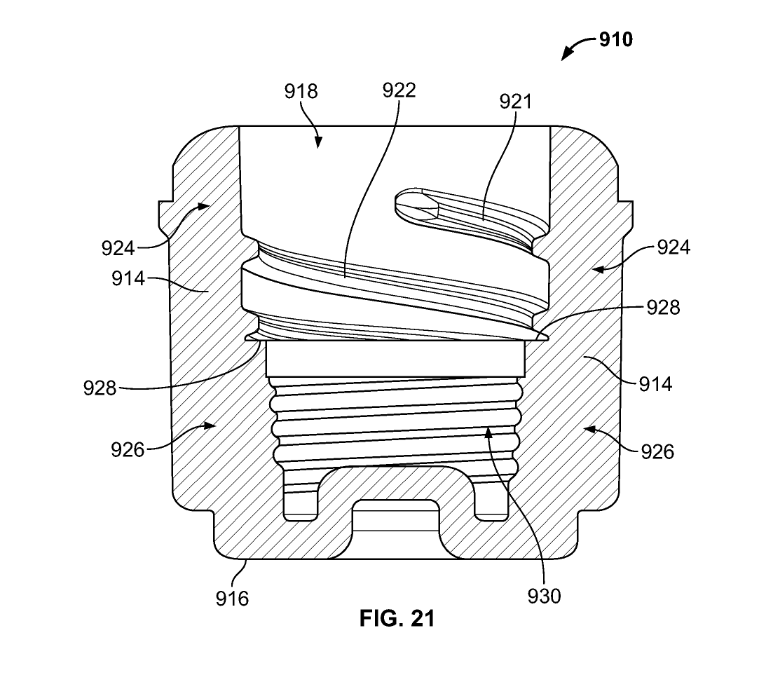

FIG. 21 is a cross-sectional view of the disinfectant cap 910 of FIG. 19. The sidewall 914 includes an upper portion 924 and a lower portion 926, where the wall of the upper portion 924 is thinner than that of the lower portion 926, thereby defining an annular ledge 928.

The inner surface of the upper portion 924 of the disinfectant cap 910 includes variable pitch threads 921. For the disinfectant cap 910, the threads 921 (e.g., double threads) start out at a constant pitch (e.g., starting pitch), such as for an approximate ¼-¾ of a turn, and then change pitch thereafter. The starting pitch is that of a standard luer lock design to assist the user with applying the disinfectant cap 910 to an LAD (e.g., relatively low torque). By varying the pitch (e.g., by reducing or increasing the pitch), as the disinfectant cap 910 is threaded onto the LAD (or other medical device), the consistent standard threads of the medical device wedge with the mismatched variable pitch threads 921 of the disinfectant cap 910.

Wedging the threads together in this fashion provides a more secured connection (e.g., from unthreading or loosening from incidental handling or contact) because a higher removal torque is needed to disengage the disinfectant cap 910 from the LAD. This design allows for engagement of the disinfectant cap 910 onto a threaded medical device (e.g., luer threads), with no other engagement mechanism (e.g., luer taper). Without a luer taper, the proposed reducing pitch threads 921 provide a secure fit than traditional luer threads alone. To create and maintain the wedge, the disinfectant cap 910 could be made of a variety of materials, such as a hard plastic (e.g., high-density polyethylene (HDPE)). LADs usually have ACME profiled threads or modified ACME profiled threads (e.g., stub ACME threads), which are known for power transmission applications due to the flat sides which distribute stress well over the faces of the thread. As a result, the ACME type threads transmit high torques while minimizing stress, which translates to better wedging action and higher removal torques.

Further, the inner diameter of the side wall 914 of the disinfectant cap 910 (e.g., major and/or minor inner diameters of the threads) can reduce in diameter further into the disinfectant cap 910. This can provide an interference fit with the outer diameter (e.g., the threads) on the medical implement. Additionally, the start of the threads 912 could be offset from the top opening 918. This could require partial insertion of the luer access device into the disinfectant cap 910, which provides alignment of the disinfectant cap 910 and luer access device before threading and facilitates threading of the disinfectant cap 910 onto the luer access device.

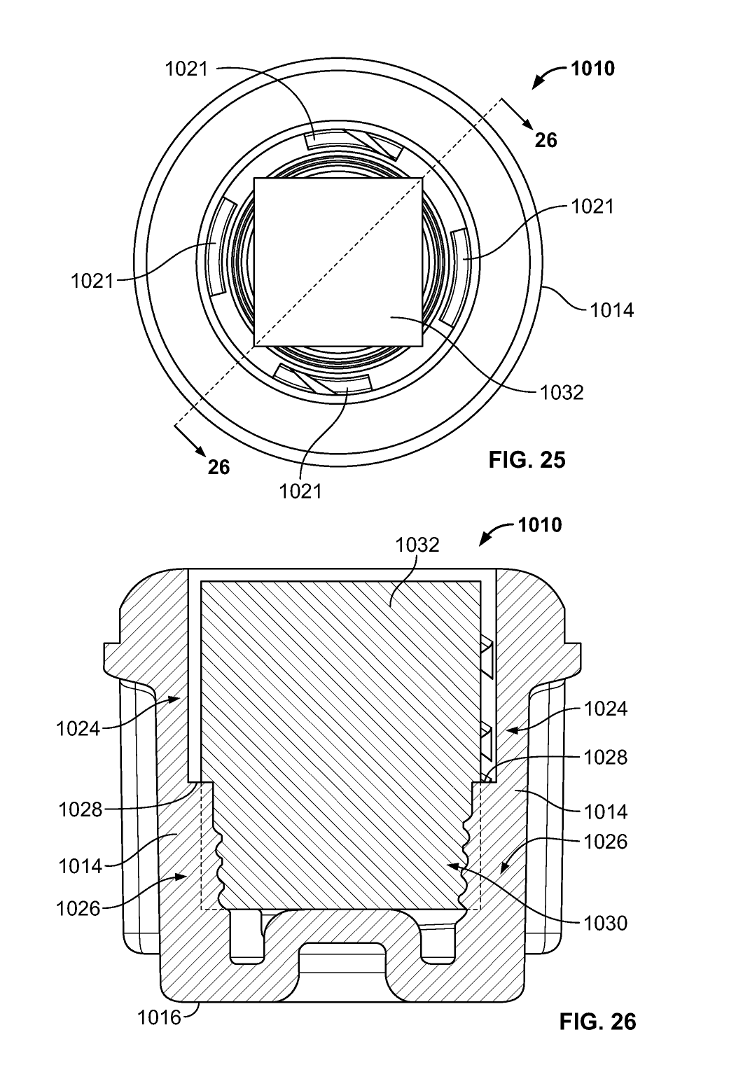

The lower portion 926 of the inner surface of the sidewall 914 includes retaining rings or threads 930, which is discussed in more detail below with respect to FIGS. 25-26.

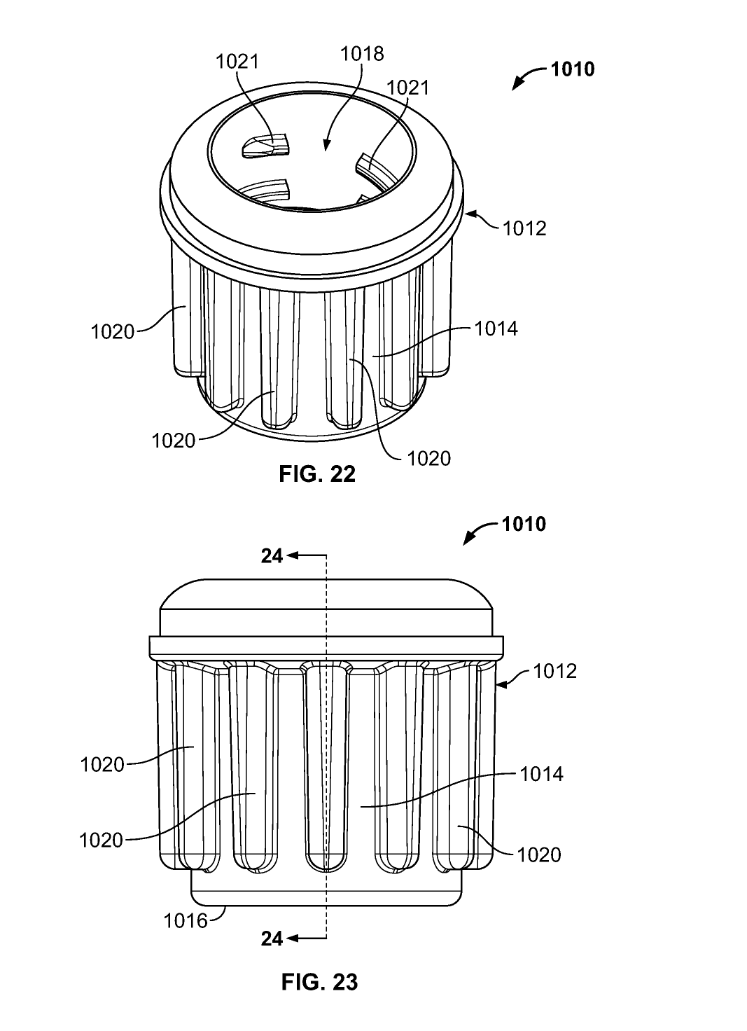

FIGS. 22-26 are views of a disinfectant cap 1010 with inner variable thread segments 1021. More specifically, FIG. 22 is a perspective view of a disinfectant cap 1010 with inner variable thread segments 1021, and FIG. 23 is a side view of the disinfectant cap 1010 of FIG. 22. Although variable thread segments 1021 are discussed with respect to a disinfectant cap 1010, the variable thread segments 1021 could be used for any threaded medical device (e.g., any threaded luer access device). Further, the variable threads shown could be used with any of the embodiments discussed above.

The disinfectant cap 1010 includes a cap body 1012 with a sidewall 1014 and a bottom wall 1016 defining an interior. The top of the sidewall 1014 defines a top opening 1018. Although not shown, a removable film could be provided over the top opening 1018. The outer surface of the sidewall 1014 includes a plurality of annularly spaced vertical ribs 1020 oriented along the axis of the disinfectant cap 1010. The vertical ribs 1020 facilitate gripping and twisting of the cap 1010 by a user. The inner surface of the sidewall 1014 includes one or more variable thread segments 1021.

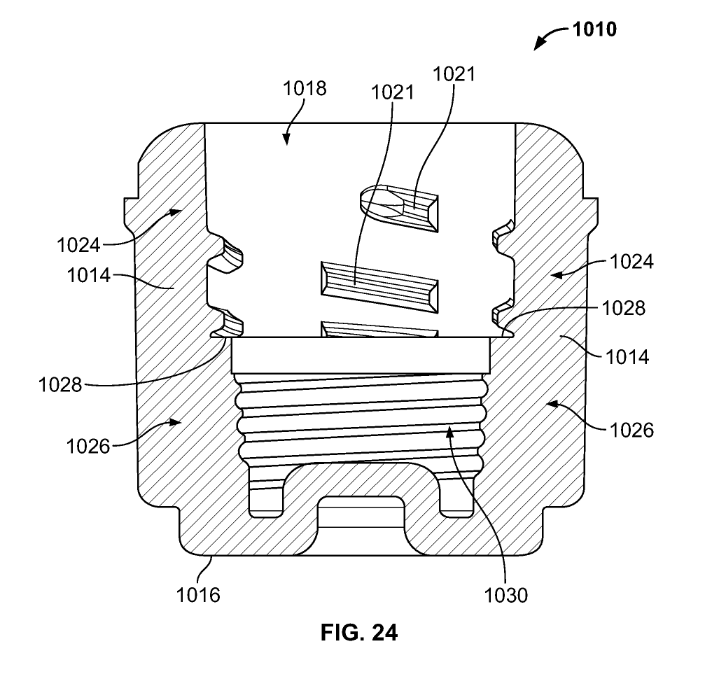

FIG. 24 is a cross-sectional view of the disinfectant cap 1010 of FIG. 22. The sidewall 1014 includes an upper portion 1024 and a lower portion 1026, where the sidewall 1014 of the upper portion 1024 is thinner than that of the lower portion 1026, thereby defining an annular ledge 1028.

The inner surface of the upper portion 1024 of the disinfectant cap includes variable pitch thread segments 1021 (e.g., helical threads with one or more breaks or interruptions). The variable thread segments 1021 operate similarly to the threads of the disinfectant cap of FIGS. 19-23, such that the variable thread segments 1021 start out at a constant pitch (e.g., starting pitch) and then change pitch thereafter. Further, the breaks between the thread segments 1021 could provide manufacturing advantages. For example, the breaks allow for a corepin tooling designs with slides to release the threads by turning ninety degrees and then pulling out. Additionally, the breaks between the thread segments 1021 allows for the sidewalls 1014 of the disinfectant cap 1010 to more easily expand or stretch if the corepin is pulled directly out during demolding.

The lower portion 1026 of the inner surface of the sidewall 1014 includes retaining rings 1030, which is discussed in more detail below with respect to FIGS. 25-26.

FIG. 25 is a top view of the disinfectant cap 1010 of FIG. 22 with a pre-soaked absorbent material 1032 inserted therein, and FIG. 26 is a cross-sectional view of the disinfectant cap 1010 of FIG. 25 with the pre-soaked absorbent material 1032 inserted therein. The lower portion 1026 of the disinfectant cap 1010 could include retaining rings 1030 (e.g., threads, protrusions, etc.) to better retain the absorbent material 1032 (e.g., sponge, pad, cellulose type pad, etc.) therein. As the disinfectant cap 1010 is being assembled, the absorbent material 1032 is positioned within the lower portion 1026 and then wetted and saturated with an antiseptic fluid, so that it expands and wedges itself therein. More specifically, the expanded absorbent material 1032 causes an interlocking effect with the retaining rings 1030, thereby preventing it from falling out of the disinfectant cap 1010, such as when the disinfectant cap 1010 is held upside down just before or after removal of the disinfectant cap from the LAD (or other medical device). Alternatively, the absorbent material 1032 could be wetted prior to assembly with the disinfectant cap 1010. An absorbent material 1032 (e.g., absorbent pad) could be retained within the disinfectant cap 1010 through other methods such as by adhesive, hot-melt, ultrasonically welding, etc.

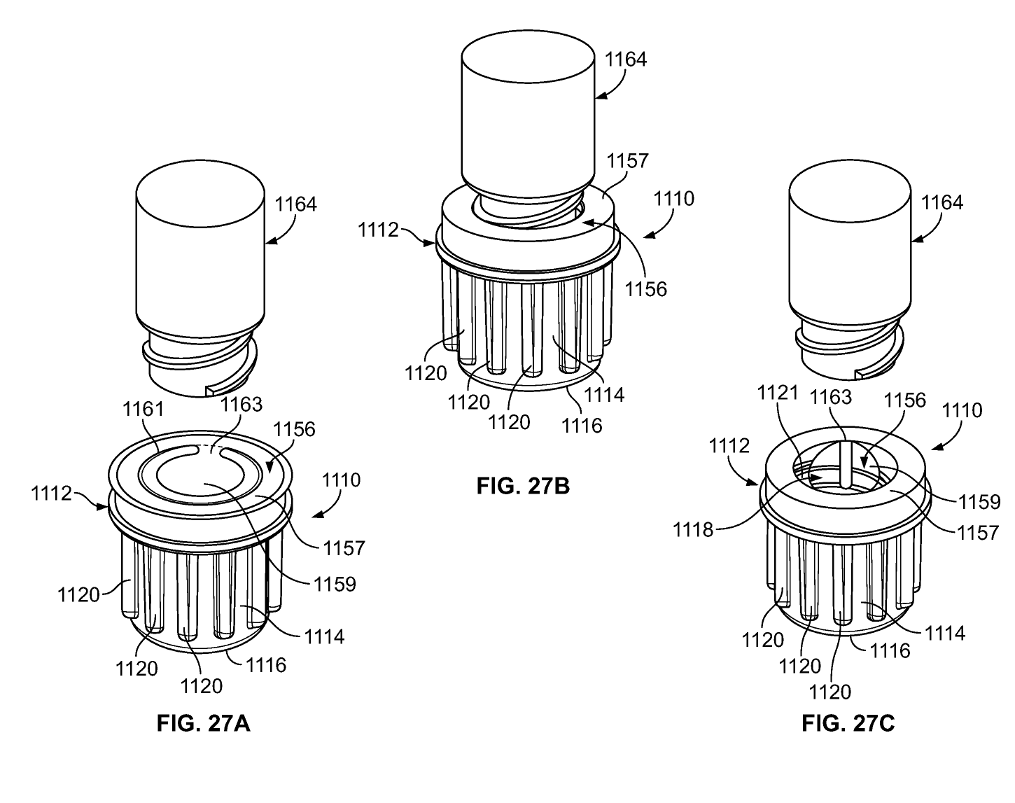

FIGS. 27A-27C are perspective views of a disinfectant cap sealed by a film with a scored area. Like other embodiments discussed above (e.g., the embodiment of FIGS. 19-26), the disinfectant cap 1110 includes a cap body 1112 with a sidewall 1114 and a bottom wall 1116 defining an interior. The top of the sidewall 1114 defines a top opening 1118. The outer surface of the sidewall 1116 could include a plurality of annularly spaced vertical ribs 1120 oriented along the axis of the disinfectant cap 1110. The inner surface of the sidewall 1114 could include one or more variable threads 1121 (and/or standard threads and/or other attachment means including snaps, interferences, etc.). The disinfectant cap 1110 can be stored in a second component, which can serve as an applicator and/or as packaging. The vertical ribs 1120 can provide rotational lock between the disinfectant cap 1110 and the second component (e.g., applicator). Lidstock can be applied to the second component (e.g., applicator) to provide a fully sterile packaging for the disinfectant cap 1110.

A film 1156 could be provided over the top opening 1118 to seal antiseptic material (e.g., antiseptic liquid, antiseptic fluid, etc.) within the antiseptic cap 1110. The film 1156 could have an outer base 1157 and an inner flap 1159, which are separable from one another by a scored area 1161 but remain connected to one another by an attached area 1163. The scored area 1161 could be perforated or otherwise weakened (e.g., in a generally circular shape) to facilitate separating the inner flap 1159 from the outer base 1157, except at the attached area 1163 (e.g., the gap in the generally circular shape of the scored area 1161). At the attached area 1163, the inner flap 1159 remains attached to the outer base 1157. Accordingly, the scored area 1161 is in a “C” shape and is not cut at a full 360 degrees in order to retain a point of attachment between the outer base 1157 and the inner flap 1159 (e.g., the attached area 1163).

As shown in FIG. 27A, the outer base 1157 and the inner area 1159 are coplanar and connected to one another by the scored area 1161 and the attached area 1163. In FIG. 27B, the medical implement 1164 (e.g., LAD) is inserted into the antiseptic cap 1110 by puncturing the film 1156. More specifically, as the medical implement 1164 contacts the film 1156 and is inserted into the top opening 1118 of the disinfectant cap 1110, the inner flap 1159 separates from the outer base 1157 along the scored area 1161, and folds downwardly (e.g., into the interior of the disinfectant cap 1110) about the attached area 1163 (which acts as a hinge). In FIG. 27C, once the medical implement 1164 is disinfected and ready for reuse, the disinfectant cap 1110 is removed from the medical implement 1164, and the film remains intact (e.g., by the attached area 1163).

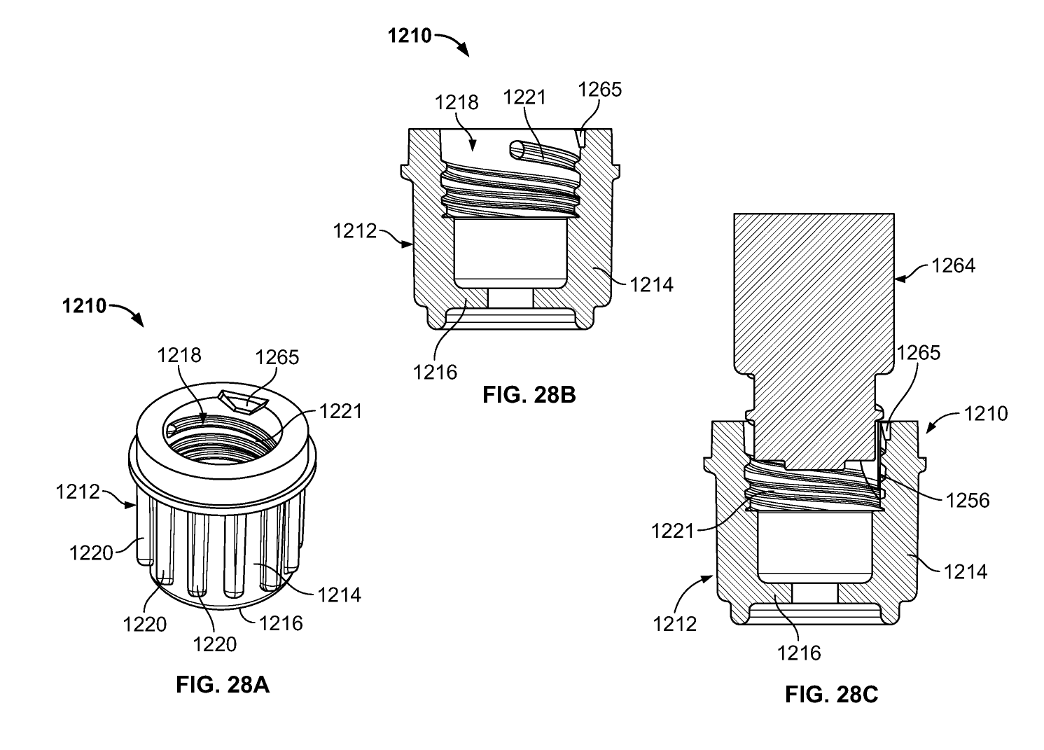

FIGS. 28A-28C are perspective views of a disinfectant cap with a notch for receiving an attached area of a film attached to a disinfectant cap. Like the embodiment of FIGS. 27A-27C discussed above, the disinfectant cap 1210 includes a cap body 1212 with a sidewall 1214 and a bottom wall 1216 defining an interior. The top of the sidewall 1214 defines a top opening 1218. The outer surface of the sidewall 1216 could include a plurality of annularly spaced vertical ribs 1220 oriented along the axis of the disinfectant cap 1210. The inner surface of the sidewall 1214 includes one or more variable threads 1221 (and/or standard threads). The disinfectant cap 1210 can be stored in a second component, which can serve as an applicator and/or as packaging. The vertical ribs 1220 can provide rotational lock between the disinfectant cap 1210 and the second component (e.g., applicator). Lidstock can be applied to the second component (e.g., applicator) to provide a fully sterile packaging for the disinfectant cap 1210.

A film 1256 could be provided over the top opening 1218, to seal antiseptic material (e.g., antiseptic liquid, antiseptic fluid, etc.) within the antiseptic cap 1210, as discussed in FIGS. 27A-27C. The film 1256 could have an outer base, an inner flap, scored area, and attached area, as discussed above. However, the scored area is optional and not required.

As shown in FIG. 28A-28B, the disinfectant cap 1210 could include a notch 1265 (or a plurality of notches) in an interior surface at the rim (e.g., at the top opening 1218) of the disinfectant cap 1210. The notch 1265 aligns with the attached area of the film 1256. In this way, as shown in FIG. 28C, when the medical device 1264 (e.g., LAD) engages the disinfectant cap 1210, the attached area folds into the notch 1265 when the inner flap separates from the outer base and folds downwardly (e.g., into the interior of the disinfectant cap 1210) about the attached area. This relieves friction and pressure on the attached area as the medical implement 1264 engages the disinfectant cap 1210, the attached area remains intact (and that the inner flap remains attached to the outer base at the attached area). The notch 1265 also provides a break in the edge of the cap 1210, along which a shearing force is created on the film (e.g., lidstock) as the LAD is inserted into the cap 1210. This break causes the film to shear around the entire circumference of the film, except at the notch 1265, thus creating the attached area and preventing the film from fully separating from the cap 1210.

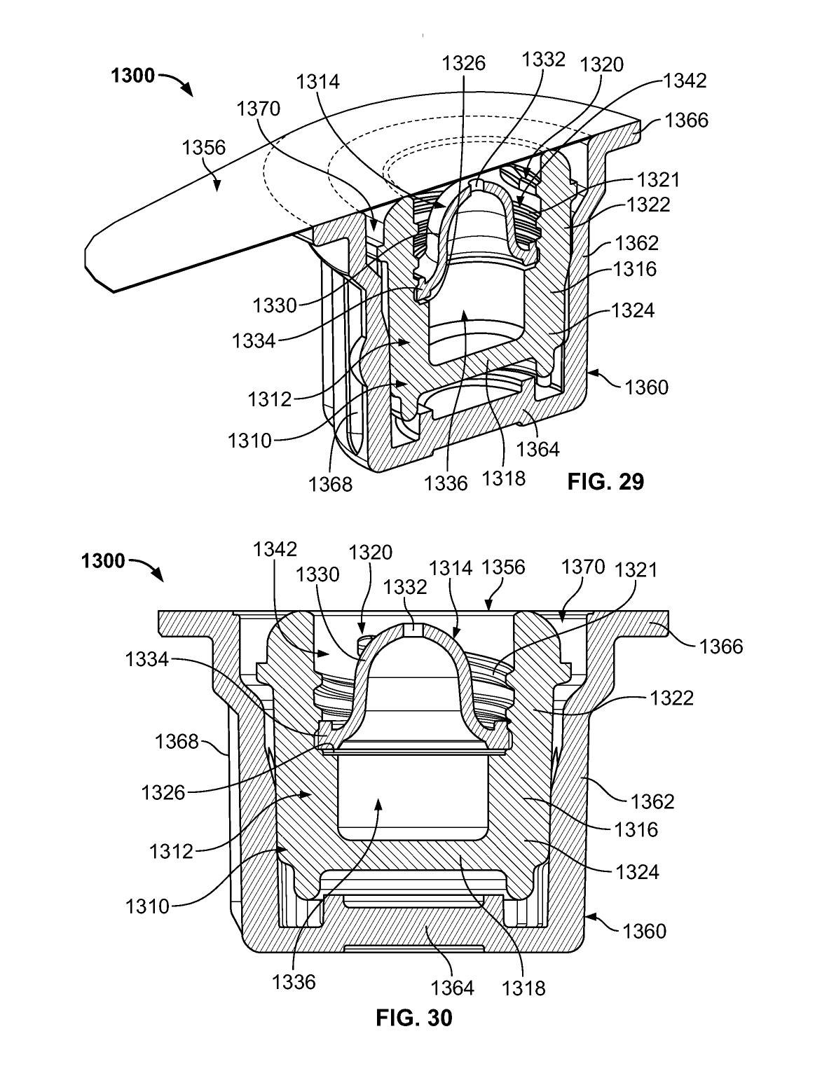

FIGS. 29-30 are views of a disinfectant cap with a dome insert sealed in a cap holder. More specifically, FIG. 29 is a cross-sectional perspective view of a disinfectant cap with a dome insert sealed in a cap holder, and FIG. 30 is a cross-sectional side view of the disinfectant cap and cap holder of FIG. 29. Like the other embodiments discussed above (e.g., the embodiments of FIGS. 13-15), the disinfectant cap 1310 includes an elastomeric dome insert 1314 positioned within the interior of the cap body 1312. The elastomeric dome insert 1314 includes a dome 1330 (e.g., hemispherical wall) having a hole 1332 (e.g., slit, opening, and/or weakened area) formed through (or substantially near) the apex thereof. The size of the hole 1332 can be sized to minimize the amount of disinfectant that might leak therethrough if the cap 1320 is positioned upside down prior to insertion of the medical device (e.g., LAD). For example, a small hole may be prone to contain the disinfectant as a liquid tends not to flow through a small hole unless pressurized (e.g., due to liquid surface tension). The elastomeric dome insert 1314 further includes an annular disc 1334 extending outwardly from the base of the dome 1330.

The disinfectant cap 1310 includes a cap body 1312 with a sidewall 1316 and a bottom wall 1318 defining an interior. The top of the sidewall 1316 defines a top opening 1320. The sidewall 1316 includes an upper portion 1322 and a lower portion 1324, with a shoulder (e.g., ledge) 1326 therebetween, such that the wall of the upper portion 1322 has a smaller thickness than the wall of the lower portion 1324. The annular disc 1334 rests on the shoulder (e.g., ledge) 1326 of the cap body 1312 when positioned therein. The lower portion 1324 of the sidewall 1316 of the cap body 1312 and the bottom surface of the elastomeric dome insert 1314 define a first chamber 1336 for containing antiseptic fluid. The upper portion 1322 of the sidewall 1316 of the cap body 1312 and the top surface of the elastomeric dome insert 1314 define a second chamber 1342 for receiving and engaging a medical device (e.g., LAD). The inner surface of the upper portion 1322 of the sidewall 1316 could include threads 1321 (e.g., variable and/or standard threads) and/or be elastically deformable to engage the LAD.

The disinfectant cap assembly 1300 includes the disinfectant cap 1310, elastomeric dome insert 1314, and cap holder 1360. The cap holder 1360 includes a sidewall 1362 and a bottom wall 1364 defining an interior. The top of the sidewall 1362 defines a top opening 1370 and includes an outwardly extending flange 1366. The outer surface of the sidewall 1316 could include a plurality of annularly spaced vertical ribs 1368 oriented along the axis of the cap holder 1360. A lidstock 1356 could be attached to the flange 1366 of the cap holder 1360 to seal the disinfectant cap 1310 therein. The top of the sidewall 1316 of the disinfectant cap 1310 could also be attached to the lidstock 1356 to seal the elastomeric dome insert 1314 and antiseptic fluid within the disinfectant cap 1310.

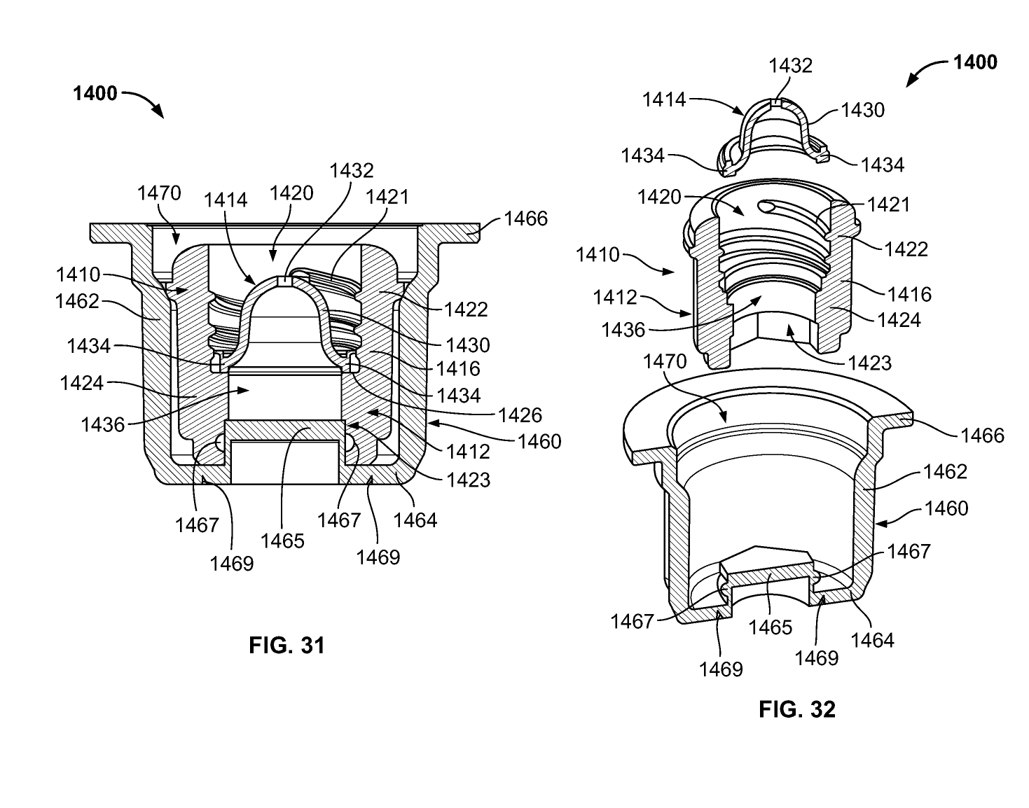

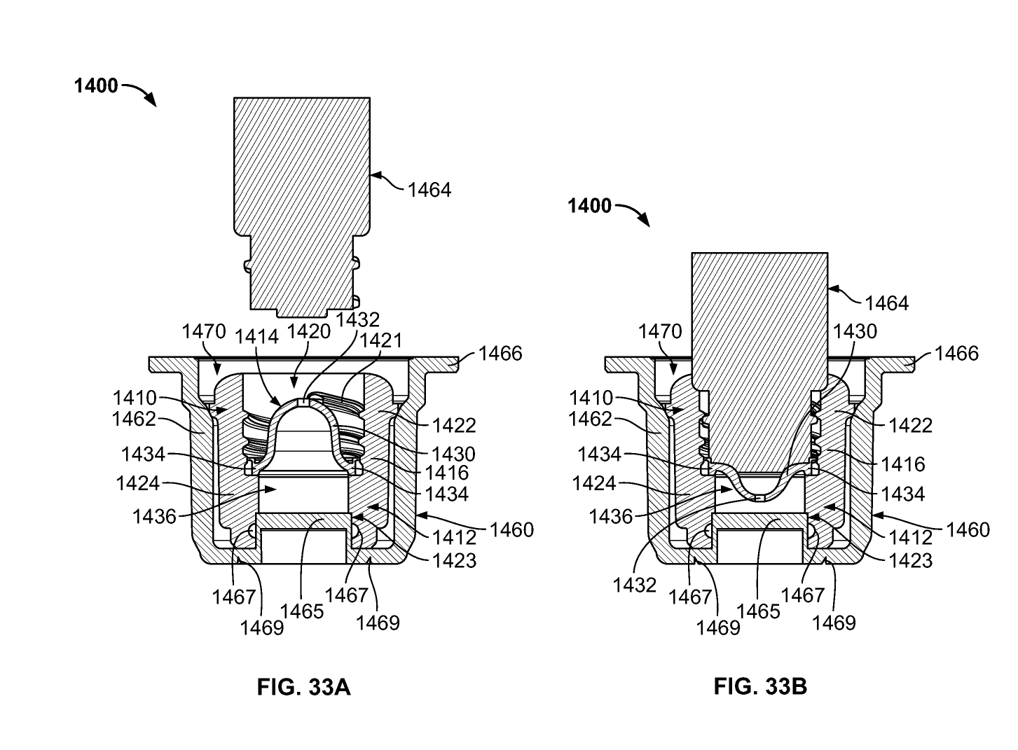

FIGS. 31-33B are views of a disinfectant cap with a dome insert sealed in a cap holder, the disinfectant cap having a bottom opening. More specifically, FIG. 31 is a cross-sectional side view of a disinfectant cap with a dome insert in a cap holder, the disinfectant cap having a bottom opening, FIG. 32 is an exploded cross-sectional perspective view of the disinfectant cap and cap holder of FIG. 31, and FIGS. 33A-33B are cross-sectional side views illustrating use of the disinfectant cap and cap holder of FIG. 31. Like the other embodiments discussed above (e.g., the embodiments of FIGS. 13-15 and 29-30), the disinfectant cap 1410 includes an elastomeric dome insert 1414 having a dome 1430 with a hole 1432. The elastomeric dome insert 1414 further includes an annular disc 1434 extending outwardly from the base of the dome 1430.

The disinfectant cap 1410 includes a cap body 1412 with a sidewall 1416 defining an interior. The top of the sidewall 1416 defines a top opening 1420, and the bottom of the sidewall 1416 defines a bottom opening 1423. The bottom opening 1423 could be hexagonally shaped (or otherwise shaped, such as circularly shaped). The sidewall 1416 includes an upper portion 1422 and a lower portion 1424, with a shoulder 1426 therebetween. The disinfectant cap assembly 1400 includes the disinfectant cap 1410, elastomeric dome insert 1414, and cap holder 1460. The cap holder 1460 includes a cap holder body 1461 with a sidewall 1462 and a bottom wall 1464 defining an interior. The top of the sidewall 1462 defines a top opening 1470 and includes an outwardly extending flange 1466. The outer surface of the sidewall 1416 could include a plurality of annularly spaced vertical ribs 1468 oriented along the axis of the cap holder 1460. As described above, a lidstock (not shown) could be attached to the flange 1466 of the cap holder 1460 to seal the disinfectant cap 1410 therein. The top of the sidewall 1416 of the disinfectant cap 1410 could also be attached to the lidstock to seal the elastomeric dome insert 1414 and antiseptic fluid within the disinfectant cap 1412.

The cap holder 1470 could further include an inwardly protruding base 1465 extending from an approximate center of the bottom wall 1464 of the cap holder 1460. The base 1465 includes one or more nubs 1467 extending from a perimeter of the base 1465. The base 1465 is approximately sized to that of the bottom opening 1423 of the disinfectant cap 1410 to be received therein. The sides of the base 1465 and/or the one or more nubs 1467 frictionally engage and/or partially deform the walls forming the bottom opening 1423 to secure the disinfectant cap 1410 to the cap holder 1460.

The lower portion 1424 of the sidewall 1416 of the cap body 1412, the bottom surface of the elastomeric dome insert 1414, and the top surface of the cap holder base 1465 define a first chamber 1436 for containing antiseptic fluid. The upper portion 1422 of the sidewall 1416 of the cap body 1412 and the top surface of the elastomeric dome insert 1414 define a second chamber 1442 for receiving and engaging a medical device (e.g., LAD). The inner surface of the upper portion 1422 of the sidewall 1416 could include threads 1421 (e.g., variable and/or standard threads) and/or be elastically deformable to engage the LAD.

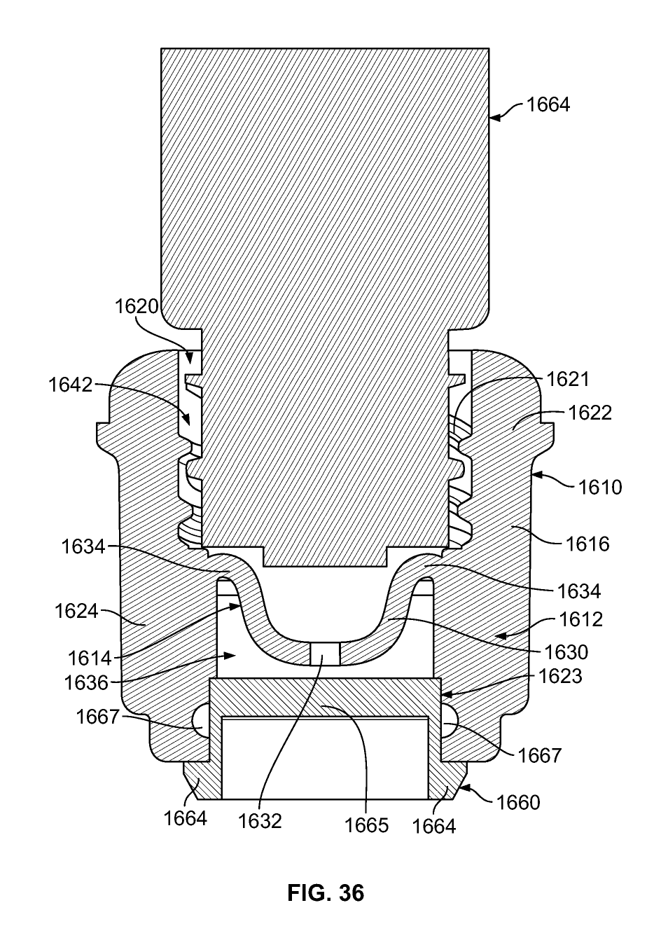

Further, the bottom wall 1464 could include one or more annular cuts 1469 (e.g., or thin webbing, or other weakened area) therein generally surrounding the inwardly protruding base 1465. Accordingly, once a user fully threads the cap 1410 and cap holder 1460 onto a medical device, the user can then continue to twist, and break away the inwardly protruding base 1465 from the rest of the cap holder 1460 along the one or more cuts 1469. This leaves the inwardly protruding base 1465 still fully engaged with the cap 1410, like the cap shown in FIG. 36 below, where the cap 1410 remains engaged with the medical device. The cap 1410 can then be unscrewed to use the medical device.

As shown in FIGS. 33A-33B, antiseptic fluid is retained within the first chamber 1436. As the disinfectant cap 1410 and cap holder 1460 engage the medical device 1464, the elastically deformable insert 1414 inwardly deforms increasing pressure of the antiseptic fluid (e.g., by decreasing the volume of first chamber 1436), and antiseptic fluid then flows onto the medical device 1464.

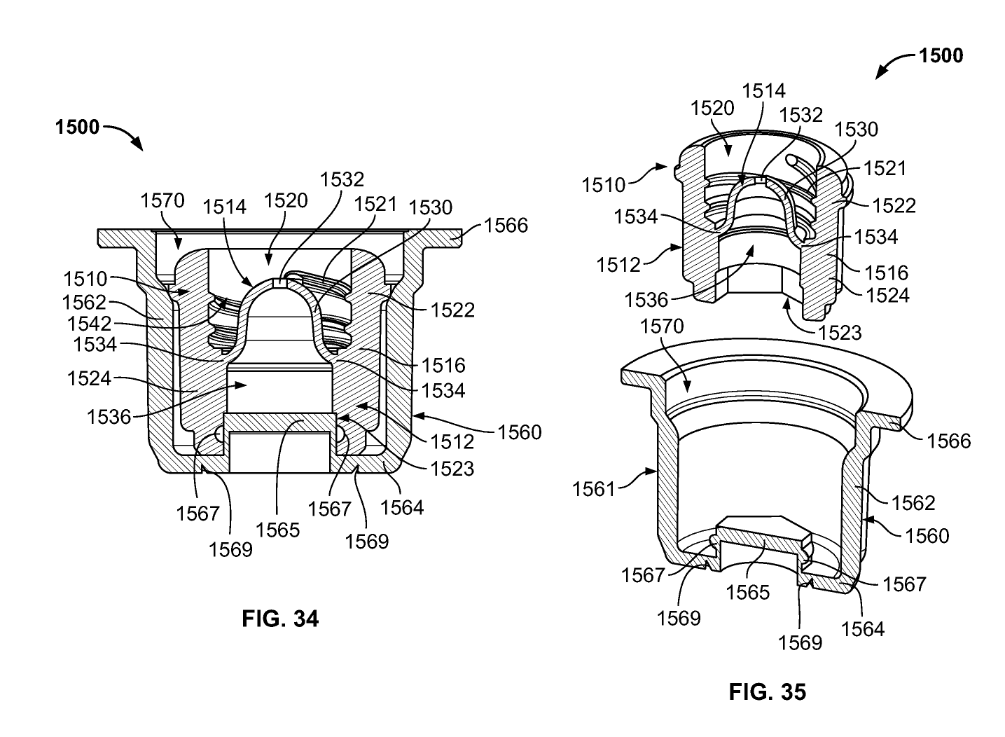

FIGS. 34-35 are views of a disinfectant cap with an integrally formed internal dome barrier in a cap holder, the disinfectant cap having a bottom opening. More specifically, FIG. 34 is a cross-sectional side view of a disinfectant cap with an integrally formed internal dome barrier in a cap holder, the disinfectant cap having a bottom opening, and FIG. 35 is an exploded cross-sectional perspective view of the disinfectant cap and cap holder of FIG. 34.

The embodiment of FIGS. 34-35 is like that of FIGS. 31-33B. The disinfectant cap 1510 includes a cap body 1512 with a sidewall 1516 defining an interior. The top of the sidewall 1516 defines a top opening 1520, and the bottom of the sidewall 1516 defines a bottom opening 1523 (e.g., hexagonally shaped). The sidewall 1516 includes an upper portion 1522 and a lower portion 1524. The inner surface of the upper portion 1522 of the sidewall 1516 could include threads 1521 (e.g., variable and/or standard threads) and/or be elastically deformable to engage the LAD.

The disinfectant cap assembly 1500 includes the disinfectant cap 1510 and cap holder 1560. The cap holder 1560 includes a cap holder body 1561 with a sidewall 1562 and a bottom wall 1564 defining an interior. The top of the sidewall 1562 defines a top opening 1570 and includes an outwardly extending flange 1566. The outer surface of the sidewall 1516 could include a plurality of annularly spaced vertical ribs (not shown). As described above, a lidstock (not shown) could be attached to the flange 1566 of the cap holder 1560 to seal the disinfectant cap 1510 therein. The top of the sidewall 1516 of the disinfectant cap 1510 could also be attached to the lidstock to seal the elastomeric dome barrier 1514 and antiseptic fluid within the disinfectant cap 1512. The cap holder 1570 could further include an inwardly protruding base 1565 extending from an approximate center of the bottom wall 1564 of the cap holder 1560. The base 1565 includes one or more nubs 1567 extending from a perimeter of the base 1565.

In this embodiment, the disinfectant cap 1510 includes an elastomeric dome barrier 1514 integrally formed with and attached to the body 1512 of the disinfectant cap 1510. The elastomeric dome barrier 1514 has a dome 1530 with a hole 1532 at (or proximate to) the apex thereof. The base 1534 of the elastomeric dome barrier 1514 is integrally attached to the interior surface of the sidewalls 1516 of the disinfectant cap 1510. Accordingly, the lower portion 1524 of the sidewall 1516 of the cap body 1512, the bottom surface of the elastomeric dome barrier 1514, and the top surface of the cap holder base 1565 define a first chamber 1536 for containing antiseptic fluid. The upper portion 1522 of the sidewall 1516 of the cap body 1512 and the top surface of the elastomeric dome barrier 1514 define a second chamber 1542 for receiving and engaging a medical device (e.g., LAD).

Further, the bottom wall 1564 could include one or more annular cuts 1569 (e.g., or thin webbing, or other weakened area) therein generally surrounding the inwardly protruding base 1565. Accordingly, once a user fully threads the cap 1510 and cap holder 1560 onto a medical device, the user can then continue to twist, and break away the inwardly protruding base 1565 from the rest of the cap holder 1560 along the one or more cuts 1569. This leaves the inwardly protruding base 1565 still fully engaged with the cap 1510, like the cap shown in FIG. 36 below, where the cap 1510 remains engaged with the medical device. The cap 1510 can then be unscrewed to use the medical device.

Alternately, the first chamber 1536 could be formed by the bottom surface of the dome barrier 1514, the sidewall 1516 of the cap body 1512, and an additional piece (not shown) that is inserted into the opening 1523. This allows the formation of the first chamber 1536 without using a surface of the cap holder 1560. This also allows the entire cap 1510 to set within the sterile packaging formed by the cap holder 1560 and the lidstock.

FIG. 36 is a cross-sectional view of a disinfectant cap with a bottom plug. The disinfectant cap of FIG. 36 is like the disinfectant cap of FIGS. 34-35. The disinfectant cap 1610 includes a cap body 1612 with a sidewall 1616 defining an interior. The top of the sidewall 1616 defines a top opening 1620, and the bottom of the sidewall 1616 defines a bottom opening 1623 (e.g., hexagonally shaped). The sidewall 1616 includes an upper portion 1622 and a lower portion 1624. The inner surface of the upper portion 1622 of the sidewall 1616 could include threads 1621 (e.g., variable and/or standard threads) and/or be elastically deformable to engage the LAD. The disinfectant cap 1610 includes an elastomeric dome barrier 1614 integrally formed with and attached to the body 1612 of the disinfectant cap 1610. The elastomeric dome barrier 1614 has a dome 1630 with a hole 1632 at (or proximate to) the apex thereof. The base 1634 of the elastomeric dome barrier 1614 is integrally attached to the interior surface of the sidewalls 1616 of the disinfectant cap 1610. As described above, a lidstock (not shown) could be attached to the disinfectant cap 1610.

The disinfectant cap 1610 includes a plug 1660. The plug 1660 includes an inwardly protruding base 1665 and outwardly extending flanges 1667. The base 1665 is approximately sized to that of the bottom opening 1623 of the disinfectant cap 1610 to be received therein. The sides of the base 1665 and/or the one or more nubs 1667 frictionally engage and/or partially deform the walls forming the bottom opening 1623 to secure the plug 1660 to the disinfectant cap 1610. The flanges 1667 prevent over insertion of the plug 1660 into the disinfectant cap 1610. Accordingly, the lower portion 1624 of the sidewall 1616 of the cap body 1612, the bottom surface of the elastomeric dome barrier 1614, and the top surface of the plug 1660 define a first chamber 1636 for containing antiseptic fluid. The upper portion 1622 of the sidewall 1616 of the cap body 1612 and the top surface of the elastomeric dome barrier 1614 define a second chamber 1642 for receiving and engaging a medical device 1664 (e.g., LAD).

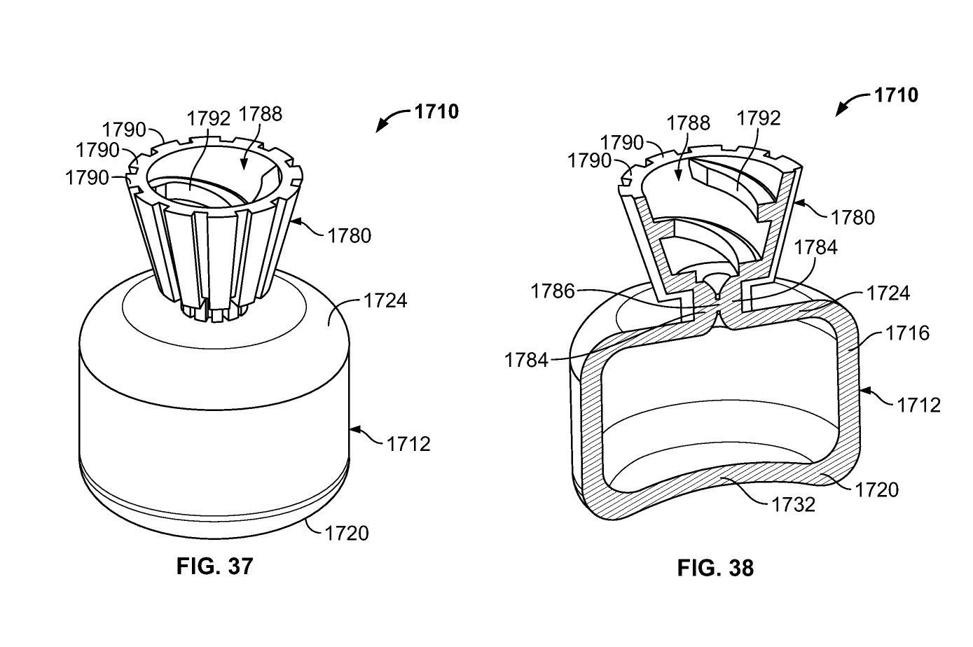

FIGS. 37-38 are views of a disinfectant cap with a frangible neck. More specifically, FIG. 37 is a perspective view of a disinfectant cap with a frangible neck, and FIG. 38 is a cross-sectional perspective view of the disinfectant cap of FIG. 37. The disinfectant cap 1710 includes a cap body 1712 with a sidewall 1716, a bottom wall 1720, and top wall 1724 defining an interior. The bottom wall 1720 of the cap body 1712 can include an invertible wall 1732 (as described in more detail above).

Extending from the top wall of the disinfectant cap is a neck 1784 which has been crimped (e.g., heat crimped) closed to form a frangible stop 1786. Extending from the neck 1784 is a receptacle 1780 defining a top opening 1788 and conical interior (e.g., tapered interior) to engage a medical device (e.g., LAD). The receptacle 1780 is of a generally inverted cone such that the top of the cone 1780 is wider than the base (e.g., where the receptacle 1780 attaches to the neck 1784). The inner surface of the receptacle 1780 includes one or more threads 1792 (variable and/or standard threads). The exterior surface of the receptacle 1780 could include a plurality of annularly spaced vertical ribs 1790. In this way, the antiseptic cap 1710 could be a one piece construction (e.g., all of the components are integrally connected to one another). A lidstock (not shown) could be attached to the top opening 1788 of the receptacle 1780.

As the medical device is inserted (e.g., threadably inserted) into the receptacle 1780, the taper of the receptacle 1780 forces the head 1780 apart (e.g., the narrowest point of the conical head 1780 closest to the neck 1784 is widened). As a result, the frangible stop 1786 is eventually forced open, and the antiseptic fluid contained within the body 1712 of the disinfectant cap 1710 flows out of the disinfectant cap 1710 through the neck 1786.

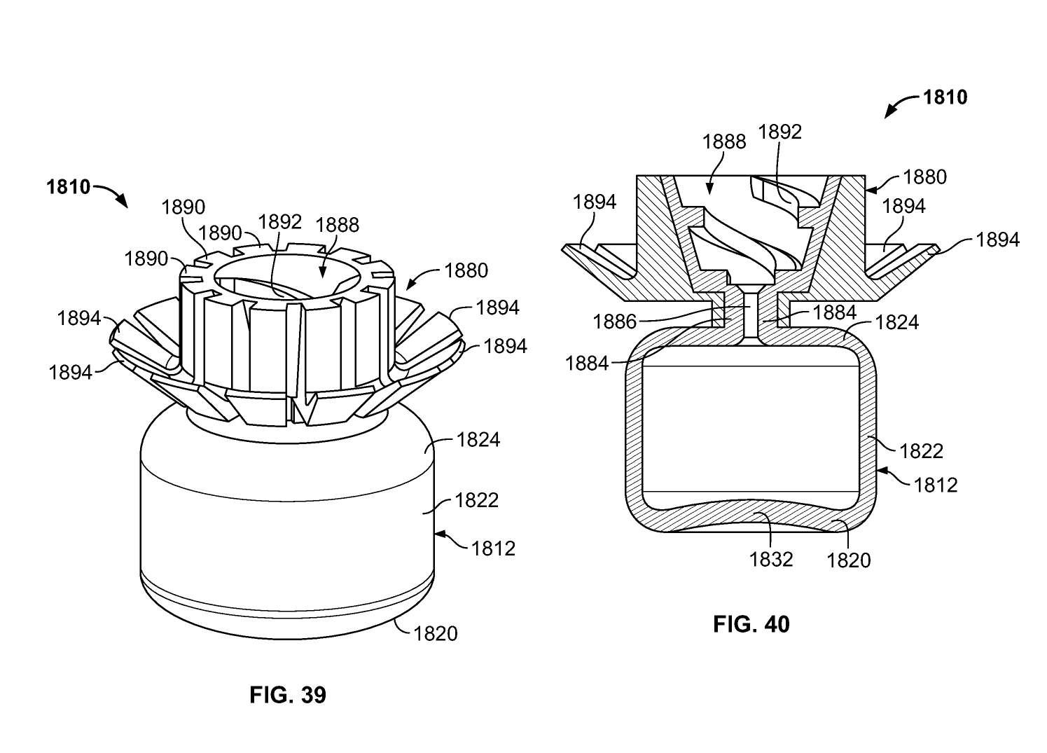

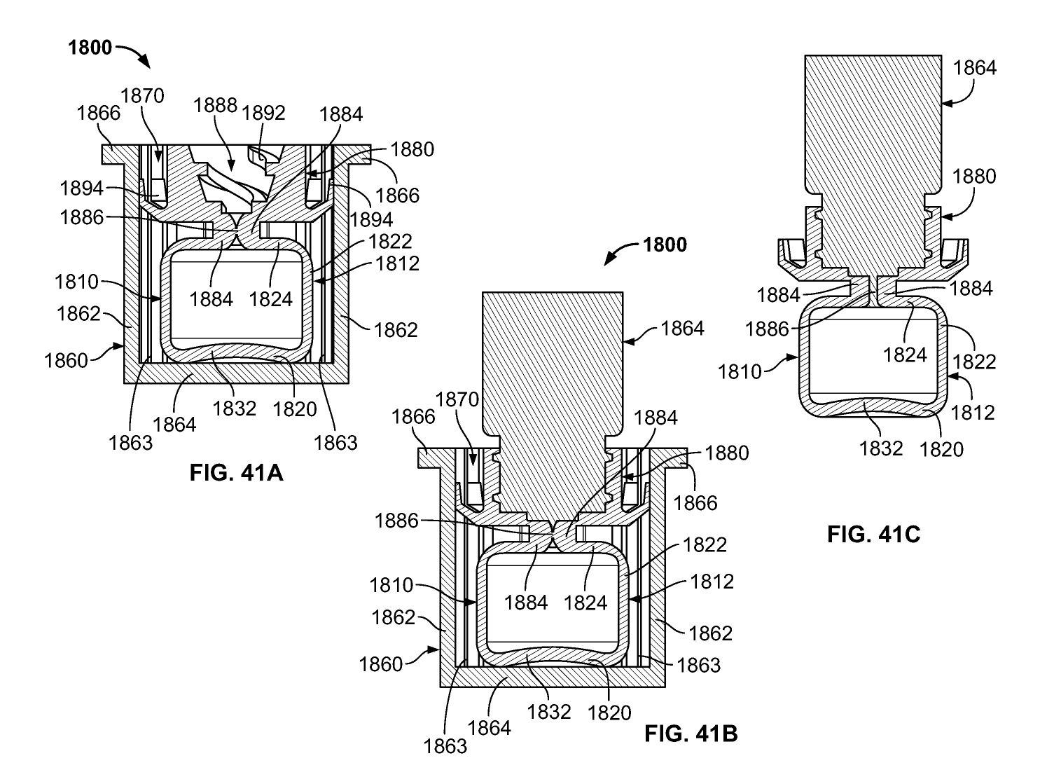

FIGS. 39-41C are views of a disinfectant cap with a frangible neck and angled fingers. More specifically, FIG. 39 is a perspective view of a disinfectant cap with a frangible neck and angled fingers, FIG. 40 is a cross-sectional perspective view of the disinfectant cap of FIG. 39, and FIGS. 41A-41C are cross-sectional side views illustrating use of the disinfectant cap of FIG. 39 and cap holder. The disinfectant cap of FIGS. 39-41C is like the disinfectant cap of FIGS. 37-38.

The disinfectant cap 1810 includes a cap body 1812 with a sidewall 1816, a bottom wall 1820, and top wall 1824 defining an interior. The bottom wall 1820 of the cap body 1812 can include an invertible wall 1832 (as described in more detail above). Extending from the top wall of the disinfectant cap is a neck 1884 which forms a channel 1886 prior to being crimped close to form a frangible stop. Extending from the neck 1884 is a receptacle 1880 defining a top opening 1888 and generally conical interior to engage a medical device (e.g., LAD). The conical interior is of a generally inverted cone such that the top of the cone 1880 is wider than the base (e.g., where the receptacle 1880 attaches to the neck 1884). The inner surface of the receptacle 1880 includes one or more threads 1892 (variable and/or standard threads). The exterior surface of the receptacle 1880 could include a plurality of annularly spaced vertical ribs 1890. In this way, the antiseptic cap 1810 could be a one piece construction (e.g., all of the components are integrally connected to one another). The receptacle 1880 further includes one or more annularly spaced upwardly angled fingers 1894 extending from a base of the receptacle 1880.

As shown in FIG. 41A, the disinfectant cap 1810 could be used with a cap holder 1860. More specifically, the disinfectant cap assembly 1800 includes the disinfectant cap 1810 and cap holder 1860. The cap holder 1860 includes a sidewall 1862 and a bottom wall 1864 defining an interior. The top of the sidewall 1862 defines a top opening 1870 and includes an outwardly extending flange 1866. A lidstock (not shown) could be attached to the flange 1866 of the cap holder 1860 to seal the disinfectant cap 1810 therein. The top of the receptacle 1880 of the disinfectant cap 1810 could also be attached to the lidstock. The outer surface of the sidewall 1816 could include a plurality of annularly spaced vertical ribs (not shown) oriented along the axis of the cap holder 1860. The interior surface of the sidewall 1816 could include a plurality of annularly spaced vertical ribs 1863.

As shown, the outermost diameter formed by the angled fingers 1894 could be greater than the inner diameter of the cap holder 1860. As a result, radial forces of the angled fingers 1894 against the cap holder keep the neck 1884 closed (thereby retaining the antiseptic fluid therein). Further, the width of the angled fingers 1894 could be approximately the same width as the spacing between the plurality of vertical ribs 1863.

As shown in FIG. 41B, as the medical device 1864 is inserted (e.g., threadably inserted) into the receptacle 1880, the base of the head 1880 (e.g., the narrowest point of the conical head 1880 closest to the neck 1884), cannot forces the head 1880 apart while it is retained within the cap holder 1860. The vertical ribs 1863 of the cap holder 1860 interact with the fingers 1894 of the disinfectant cap 1810 to facilitate threading of the disinfectant cap 1810 onto the medical device 1864 by preventing relative rotation of the disinfectant cap 1810 and cap holder 1860.

As shown in FIG. 41C, once the disinfectant cap 1810 is fully engaged with the medical device 1864, the cap holder 1860 is removed from the disinfectant cap 1810. At this point, the frangible stop 1886 is forced open and forms a channel, and the antiseptic fluid contained within the body 1812 of the disinfectant cap 1810 flows out of the disinfectant cap 1810 through the neck 1886 and onto the medical device 1864.

A number of the components and features discussed above could be integrally constructed or separately attached. For example, the disinfectant cap and cover of FIGS. 1-3 could be made of a single piece (e.g., the tapered portion being integrally formed with or as part of the outer walls of the disinfectant cap).

Having thus described the system and method in detail, it is to be understood that the foregoing description is not intended to limit the spirit or scope thereof. It will be understood that the embodiments of the present disclosure described herein are merely exemplary and that a person skilled in the art may make any variations and modification without departing from the spirit and scope of the disclosure. All such variations and modifications, including those discussed above, are intended to be included within the scope of the disclosure.

1. A disinfectant cap for disinfecting a surface of a medical device, comprising:
a cap body having a bottom wall and a sidewall extending therefrom, the sidewall defining a top opening;
antiseptic fluid contained within the cap body; and
a top wall connected to the cap body, the top wall deformable from a first position sealing the antiseptic fluid within the cap body to a second position releasing the antiseptic fluid from the cap body.

2. The disinfectant cap of claim 1, wherein the bottom wall of the cap body is inwardly deformable from a first convex orientation to a second concave orientation.

3. The disinfectant cap of claim 2, wherein the bottom wall of the cap body includes a bulb integrally formed with the cap body.

4. The disinfectant cap of claim 1, wherein the top wall comprises a cover.

5. The disinfectant cap of claim 4, further comprising a removable film attached to a top surface of the cover.

6. The disinfectant cap of claim 5, wherein the cap body further comprises a neck extending from the sidewall, the circumference of the neck being smaller than the circumference of the sidewall.

7. The disinfectant cap of claim 6, wherein the neck includes a flange extending from a top of the neck, and the cover includes a planar circular top portion with a depending rim, the depending rim including a lip extending from a bottom thereof to engage the flange of the neck of the cap body.

8. The disinfectant cap of claim 4, wherein the top wall includes a frangible tip in the first position, the top wall having an inner wall extending downwardly from a top portion of the top wall to the frangible tip.

9. The disinfectant cap of claim 8, wherein the frangible tip of the top wall is separated in the second position.

10. The disinfectant cap of claim 8, wherein the frangible tip is submerged in the antiseptic fluid contained in the cap body, and the inner wall defines a central aperture to engage a medical device.

11. The disinfectant cap of claim 1, wherein the top wall comprises an elastomeric dome including a partial slit formed therethrough, the partial slit closed when the elastomeric dome is in the first position and opened when the elastomeric dome is inverted to the second position.

12. The disinfectant cap of claim 11, wherein the elastomeric dome is attached to the top of the cap body.

13. The disinfectant cap of claim 12, wherein the elastomeric dome cover includes ribs extending around a surface thereof

14. The disinfectant cap of claim 12, wherein the elastomeric dome cover includes an outwardly extending helical rib.

15. The disinfectant cap of claim 11, wherein the elastomeric dome is recessed within the cap body.

16. The disinfectant cap of claim 15, wherein the sidewall includes a lower portion and an upper portion with a shoulder therebetween, the lower portion defining a first chamber for containing the antiseptic fluid, the upper portion defining a second chamber for receiving and engaging the medical device, and the elastomeric dome insert proximate the shoulder.

17. The disinfectant cap of claim 1, wherein the medical device is a luer access device.

18. The disinfectant cap of claim 1, wherein the antiseptic fluid comprises alcohol.

19. The disinfectant cap of claim 1, wherein the cap body further comprises internal variable threads.

20. A medical device attachment, comprising:
a body having a sidewall and a bottom wall, the body defining an interior and a top opening; and
one or more variable threads disposed on an inner surface of the sidewall, the one or more variable threads sized and positioned to be wedged with standard threads of a medical device when applied thereto.

21. The medical device attachment of claim 20, wherein the threaded medical device is a luer access device.

22. The medical device attachment of claim 20, wherein the variable threads are at a constant pitch proximate the opening and then change pitch further into an interior of the medical device attachment.

23. The medical device attachment of claim 20, wherein an inner diameter of the sidewall reduces in size further from the opening to provide an interference fit with an outer diameter of the medical device.

24. The medical device attachment of claim 20, wherein a start of the threads is offset from the opening to align the medical device attachment with the medical device before threading.

25. The medical device attachment of claim 20, further comprising a plurality of annularly spaced vertical ribs disposed on an outer surface of the sidewall to facilitate gripping and twisting of the medical device attachment.

26. The medical device attachment of claim 20, wherein the medical device attachment is a disinfectant cap.

27. The medical device attachment of claim 26, wherein the body includes an upper portion proximate the opening and a lower portion, the upper portion including the one or more variable threads, the lower portion including one or more retaining rings.

28. The medical device attachment of claim 27, further comprising an absorbent material positioned within the lower portion, the absorbent material including antiseptic fluid.

29. The medical device attachment of claim 28, further comprising a removable film over the opening.

30. A disinfectant cap for disinfecting a surface of a medical device, comprising:
a cap body having a plurality of walls defining a top opening and an interior;
antiseptic fluid sealed within the cap body, the interior of the cap body pre-pressurized to a pressure greater than atmospheric pressure, and the antiseptic fluid released from the cap body upon application of the cap body to a medical device.

31. The disinfectant cap of claim 30, wherein the plurality of walls include a bottom wall, the bottom wall inwardly deformable from a first convex orientation to a second concave orientation.

32. The disinfectant cap of claim 30, further comprising a removable film attached to the top opening.

33. The disinfectant cap of claim 30, wherein the cap body further comprises a neck extending from a sidewall, the circumference of the neck being smaller than the circumference of the sidewall.

34. The disinfectant cap of claim 33, wherein the top opening is defined by a receptacle extending from a top of the neck of the cap body.

35. The disinfectant cap of claim 34, wherein the neck of the cap body is crimped closed prior to application to a medical device, and the neck is broken open when applied to a medical device.

36. The disinfectant cap of claim 30, wherein the plurality of walls includes a top wall, the top wall comprising a cover.

37. The disinfectant cap of claim 36, wherein the top wall includes a frangible tip.

38. The disinfectant cap of claim 37, wherein the frangible tip is submerged in the antiseptic fluid contained in the cap body, and the inner wall defines a central aperture to engage a medical device.

39. The disinfectant cap of claim 38, wherein the top wall comprises an elastomeric dome including a partial slit formed therethrough, the partial slit closed when the elastomeric dome is in the first position and opened when the elastomeric dome is inverted to the second position.

40. The disinfectant cap of claim 30, wherein the plurality of walls includes a top wall, the top wall comprising an elastomeric dome including a partial slit formed therethrough.

41. The disinfectant cap of claim 40, wherein the elastomeric dome cover includes ribs extending around a surface thereof

42. The disinfectant cap of claim 40, wherein the elastomeric dome cover includes an outwardly extending helical rib.

43. The disinfectant cap of claim 30, wherein the medical device is a luer access device.

44. The disinfectant cap of claim 30, wherein the antiseptic fluid comprises alcohol.

45. The disinfectant cap of claim 30, wherein the cap body further comprises variable threads.
